(19) United States
(12) Patent Application Publication
(10) Pub. No.: US 2013/0338216 A1
(43) Pub. Date: Dec. 19, 2013

(54) DEUTERATED AND/OR FLUORINATED TAXANE DERIVATIVES

(71) Applicants: Antoni Kozlowski, Huntsville, AL (US); Timothy A. Riley, Alameda, CA (US); Samuel P. Mcmanus, Guntersville, AL (US)

(72) Inventors: Antoni Kozlowski, Huntsville, AL (US); Timothy A. Riley, Alameda, CA (US); Samuel P. Mcmanus, Guntersville, AL (US)

(73) Assignee: NEKTAR THERAPEUTICS, SAN FRANCISCO, CA (US)

(21) Appl. No.: 13/995,058

(22) PCT Filed: Dec. 22, 2011
(86) PCT No.: PCT/US11/66876

§ 371 Date: Aug. 27, 2013

Related U.S. Application Data
(60) Provisional application No. 61/426,202, filed on Dec. 22, 2010.

(51) Int. Cl. C07D 305/14

(52) U.S. Cl. CPC C07D 305/14
USPC 514/449; 549/510; 549/511

(57) ABSTRACT

The invention relates to (among other things) deuterated and/or fluorinated docetaxel and cabazitaxel and derivatives thereof, as well as compositions comprising each of the foregoing.

### CROSS REFERENCE TO RELATED APPLICATION

This application claims the benefit of priority under 35 U.S.C. §119(e) to U.S. Provisional Patent Application Ser. No. 61/426,202, filed on Dec. 22, 2010, the disclosure of which is incorporated herein by reference in its entirety.

### FIELD OF THE INVENTION

This invention comprises (among other things) deuterated and/or fluorinated taxane derivatives. The compounds described herein relate to and/or have application(s) in (among others) the fields of drug discovery, pharmacotherapy, physiology, organic chemistry and polymer chemistry.

### BACKGROUND OF THE INVENTION

Docetaxel is a taxane that is a clinically well established oncolytic agent used mainly for the treatment of breast, ovarian, and non-small cell lung cancer. Docetaxel is recommended for treatment of patients who have locally advanced, or metastatic breast or non-small cell lung cancer who have failed to stop cancer progression or relapsed following anthracycline-based chemotherapy. In Europe, docetaxel is also approved for use in certain types of prostate cancer. The structure of docetaxel is shown below.

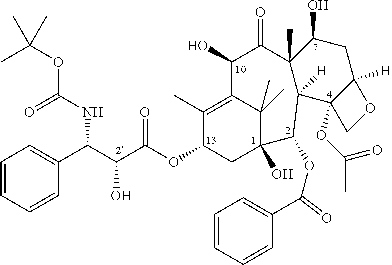

Docetaxel is mainly metabolised in the liver by the cytochrome P450 CYP3A4 and CYP3A5 subfamilies of isoenzymes. Metabolism is principally oxidative and at the t-butoxy (t-Boc) side chain, resulting first in an alcohol docetaxel (“M2”), which is then cyclised to three further metabolites (“M1,” “M3” and “M4”). The metabolites are largely inactive, which results in docetaxel suffering from not having a sufficient circulatory lifetime in vivo. The circulation lifetime can be extended by polymer conjugation to form prodrugs. See WO 2010/019233. Even when provided in the form of a prodrug, docetaxel will (following its release from the prodrug) still exhibit the basic loss of activity by these metabolic processes.

Cabazitaxel (XRP-6258) is also a drug within the taxoid class of anti-cancer agents that was more recently approved (in combination with prednisone) for the treatment of individuals suffering from hormone-refractory metastatic prostate cancer who are were previously treated with a docetaxel-containing treatment regimen. Cabazitaxel, as a microtubule inhibitor, binds to tubulin and promotes its assembly into microtubules while simultaneously inhibiting disassembly. This leads to the stabilization of microtubules, which results in the inhibition of mitotic and interphase cellular functions.

Cabazitaxel has the following chemical structure:

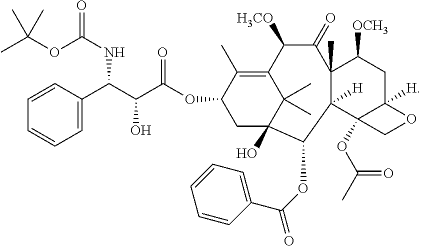

Commercially, cabazitaxel is available as the acetone solvate under the JEVTANA® brand from Sanofi-aventis (Bridgewater, N.J.). This commercially available formulation also includes Polysorbate 80 as a solublizing agent for the drug.

Like docetaxel, cabazitaxel is extensively metabolized in the liver (>95%) by the cytochrome P450 CYP3A4 and CYP3A5 subfamilies of isoenzymes

Although shown to provide several advantages over other taxanes (such as docetaxel) for certain indications, cabazitaxel is also associated with drawbacks as well. Clinically, for example, cabazitaxel is known to have a much higher toxicity than docetaxel. The toxicity is believed to be dependent on the rate of drug clearance from the body, which is largely determined by the patient's cytochrome CYP3A4 metabolic activity. In this regard, because of differences in CYP3A4 metabolic activity within the population, patients given a standard dose of cabazitaxel exhibit a wide interpatient variation in clearance and toxic effects.

Although other taxanes are available, these are believed to have even less desirable properties than docetaxel and cabazitaxel. Thus, there exists a clinical need for new taxanes which can be leveraged by the clinician to provide better targeted treatment regimens.

The present invention seeks to address these and other needs in the art.

### SUMMARY OF THE INVENTION

In one or more embodiments of the invention, a deuterated and/or fluorinated cabazitaxel is provided.

In one or more embodiments of the invention, a deuterated cabazitaxel is provided.

In one or more embodiments of the invention, a fluorinated cabazitaxel is provided.

In one or more embodiments of the invention, a deuterated and/or fluorinated docetaxel is provided.

In one or more embodiments of the invention, a deuterated docetaxel is provided.

In one or more embodiments of the invention, a fluorinated docetaxel is provided.

In one or more embodiments of the invention, a compound is provided, the compound having a structure encompassed by the following formula:

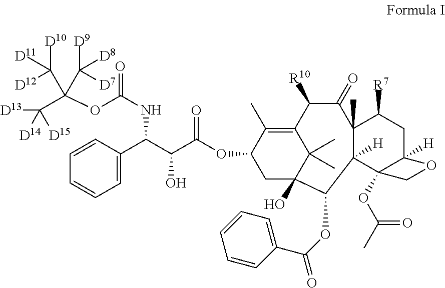

wherein:
R¹⁰ is selected from the group consisting of OH, OC₁₋₆ organic radical (e.g., methoxy), and a deuterated/fluorinated OC₁₋₆ organic radical (e.g.,

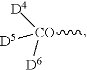

wherein D⁴ is selected from the group consisting of H, D, F and CF₃; D⁵ is selected from the group consisting of H, D, F and CF₃; and D⁶ is selected from the group consisting of H, D, F and CF₃);
R⁷ is selected from the group consisting of OH, a OC₁₋₆ organic radical (e.g., methoxy), and a deuterated/fluorinated OC₁₋₆ organic radical (e.g.,

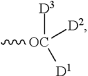

wherein D¹ is selected from the group consisting of H, D, F and CF₃; D² is selected from the group consisting of H, D, F and CF₃; and D³ is selected from the group consisting of H, D, F and CF₃);
D⁷ is selected from the group consisting of H, D and F;
D⁸ is selected from the group consisting of H, D and F;
D⁹ is selected from the group consisting of H, D and F;
D¹⁰ is selected from the group consisting of H, D and F;
D¹¹ is selected from the group consisting of H, D and F;
D¹² is selected from the group consisting of H, D and F;
D¹³ is selected from the group consisting of H, D and F;
D¹⁴ is selected from the group consisting of H, D and F; and
D¹⁵ is selected from the group consisting of H, D and F,
   and further wherein at least one atom in the compound is either D or F,
   and pharmaceutically acceptable salts thereof.

In one or more embodiments of the invention, a compound is provided, the compound having a structure encompassed by the following formula:

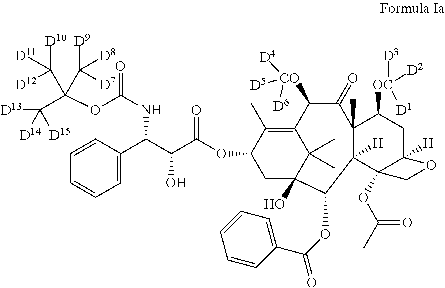

wherein:
D¹ is selected from the group consisting of H, D, F and CF₃;
D² is selected from the group consisting of H, D, F and CF₃;
D³ is selected from the group consisting of H, D, F and CF₃;
D⁴ is selected from the group consisting of H, D, F and CF₃;
D⁵ is selected from the group consisting of H, D, F and CF₃;
D⁶ is selected from the group consisting of H, D, F and CF₃;
D⁷ is selected from the group consisting of H, D and F;
D⁸ is selected from the group consisting of H, D and F;
D⁹ is selected from the group consisting of H, D and F;
D¹⁰ is selected from the group consisting of H, D and F;
D¹¹ is selected from the group consisting of H, D and F;
D¹² is selected from the group consisting of H, D and F;
D¹³ is selected from the group consisting of H, D and F;
D¹⁴ is selected from the group consisting of H, D and F; and
D¹⁵ is selected from the group consisting of H, D and F,
and further wherein at least one of D¹, D², D³, D⁴, D⁵, D⁶, D⁷, D⁸, D⁹, D¹⁰, D¹¹, D¹², D¹³, D¹⁴ and D¹⁵ is not H,
   and pharmaceutically acceptable salts thereof.

In one or more embodiments of the invention, a composition is provided, the composition comprising a deuterated and/or fluorinated taxane and an optional pharmaceutically acceptable excipient.

In one or more embodiments of the invention, a composition is provided, the composition comprising a compound encompassed within Formula I and an optional pharmaceutically acceptable excipient.

In one or more embodiments of the invention, a composition is provided, the composition comprising a compound encompassed within Formula Ia and an optional pharmaceutically acceptable excipient.

In one or more embodiments of the invention, a dosage form is provided, the dosage form comprising a deuterated and/or fluorinated taxane.

In one or more embodiments of the invention, a dosage form is provided, the dosage form comprising a compound encompassed within Formula I.

In one or more embodiments of the invention, a dosage form is provided, the dosage form comprising a compound encompassed within Formula Ia.

In one or more embodiments of the invention, a method is provided, the method comprising deuterating a taxane.

In one or more embodiments of the invention a method is provided, the method comprising fluorinating a taxane.

In one or more embodiments of the invention, a method is provided, the method comprising administering a deuterated and/or fluorinated taxane to a mammal in need thereof.

In one or more embodiments of the invention, a method is provided, the method comprising administering a compound of encompassed within Formula Ito a mammal in need thereof.

In one or more embodiments of the invention, a method is provided, the method comprising administering a compound of encompassed within Formula Ia to a mammal in need thereof.

Additional embodiments of the present compounds, compositions, methods, and the like will be apparent from the following description, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims.

### BRIEF DESCRIPTION OF THE DRAWINGS

FIGS. 1A through 1F are plots showing the cytotoxicity of docetaxel and d₉-docetaxel toward the A549 cell line (FIG. 1A and FIG. 1B, respectively), the MDA-MB-231 cell line (FIG. 1C and FIG. 1D, respectively), and the NCI-H460 cell line (FIG. 1E and FIG. 1F, respectively), a further discussed in Example 5.

FIG. 2 is a showing the body weight change (as a percentage) following intravenous administration of d₉-docetaxel, as further discussed in Example 6.

FIG. 3A and FIG. 3B are plots of the stabilities of various tested taxanes in human liver microsomes and boiled liver microsomes, respectively, as further discussed in Example 7.

### DETAILED DESCRIPTION OF THE INVENTION

As used in this specification, the singular forms “a,” “an,” and “the” include plural referents unless the context clearly dictates otherwise.

When reference is made to deuterium (“D”) and deuteration it is intended to include also tritium and tritiation, or a mixture of deuterium and tritium.

In describing and claiming the present invention, the following terminology will be used in accordance with the definitions described below.

“Substantially” or “essentially” means nearly totally or completely, for instance, 95% or greater, more preferably 97% or greater, still more preferably 98% or greater, even more preferably 99% or greater, yet still more preferably 99.9% or greater, with 99.99% or greater being most preferred of some given quantity.

“Pharmacologically effective amount,” “physiologically effective amount,” and “therapeutically effective amount” are used interchangeably herein to mean the amount of the compound of the invention present in a composition that is needed to provide a desired level of the compound (or desired metabolite thereof) in the bloodstream or in the target tissue. The precise amount may depend upon numerous factors, e.g., the particular active agent, the components and physical characteristics of the composition, intended patient population, patient considerations, and may readily be determined by one skilled in the art, based upon the information provided herein and available in the relevant literature.

A basic reactant or an acidic reactant described herein include neutral, charged, and any corresponding salt forms thereof.

The term “patient,” refers to a living organism suffering from or prone to a condition that can be prevented or treated by administration of a compound of the invention as described herein, and includes both humans and animals.

“Optional” or “optionally” means that the subsequently described circumstance may but need not necessarily occur, so that the description includes instances where the circumstance occurs and instances where it does not.

As indicated above, the present invention is directed to (among other things) a deuterated and/or fluorinated cabazitaxel. Any method for making the deuterated and/or fluorinated cabazitaxel can be used and the invention is not limited in this regard.

In some instances, it is possible to prepare the deuterated and/or fluorinated cabazitaxel by starting with a taxane and replacing hydrogen atoms for deuterium. Thus, as a starting material, cabazitaxel can be prepared synthetically as described in U.S. Pat. Nos. 5,847,170 and 5,962,705. In addition, a formulation containing cabazitaxel can be obtained under the JEVTANA® brand from Sanofi-aventis (Bridgewater, N.J.). If necessary, cabazitaxel can be separated using conventional techniques like extraction or silica gel chromatography. Similarly, docetaxel can also be prepared synthetically and obtained from commercial sources.

Having obtained a taxane as a starting material, some compounds of the invention can be prepared using exchange approaches which exchange deuterium or deuterium-containing groups for protons or proton-containing groups respectively. In this regard, a taxane (such as cabazitaxel and docetaxel) is placed into contact with a deuterium-rich environment (by, for example, placing in a deuterium-rich solvent), thereby allowing for exchange of deuterium for proton(s) within the molecule.

In addition, the compounds of the invention can be prepared using deuterated/fluorinated reagent(s) which are reagents that contain deuterium or fluorine atoms instead of hydrogen. In this regard, compounds of the invention can be prepared by (for example) following a known method for preparing a taxane except conventional reagent(s) used to synthesize the taxane are replaced with one or more deuterated/fluorinated reagents. In this way, the deuterated/fluorinated reagents can provide the desired deuterium and/or fluorine atoms within the compound and at the desired location(s). Exemplary synthetic methods for preparing cabazitaxel include those described in U.S. Pat. Nos. 5,847,170 and 5,962,705. Exemplary synthetic methods for preparing docetaxel include those described in U.S. Pat. No. 4,814,470. With regard to deuterated/fluorinated reagents, such reagents are available commercially from suppliers such as Sigma-Aldrich, St. Louis Mo., Shanghai Sinofluoro Scientific Co. Ltd., Shanghai City, Shanghai China, TCI America, Portland Oreg., and Icon Isotopes, Summit N.J. and described in “Fluorine-Containing Reagents,” Leo A. Paquette (ed.) in Handbook of Reagents, Wiley Interscience (2010) ISBN: 978-0-470-66649-4. In addition, deuterated reagents necessary to prepare compounds of the invention can be prepared via an exchange reaction.

An exemplary approach for preparing compounds of the invention includes alkylation using a deuterated/fluorinated reagent of the commercially available (e.g., from Sigma-Aldrich, St. Louis Mo.) compound 10-deacetylbaccatin III. As shown in the schematic below, the deuterated/fluorinated reagent, trideuteromethyl iodide, is used to alkylate 10-deacetylbaccatin III:

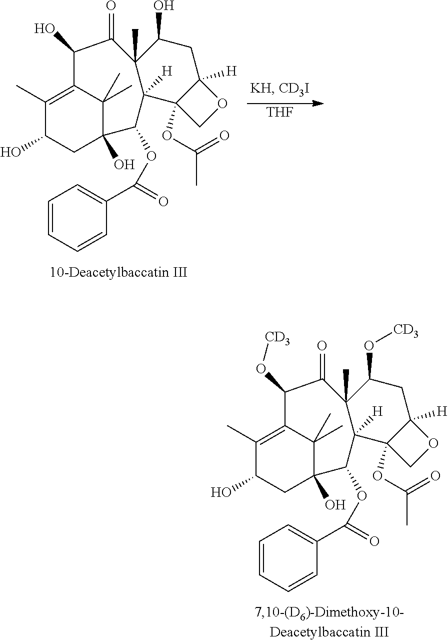

Although the schematic above used the deuterated/fluorinated reagent, trideuteromethyl iodide, other deuterated/fluorinated reagents that are alkylating agents (other “deuterated/fluorinated alkylating agents”) can be used in place of trideuteromethyl iodide. In this regard, deuterated alkyl halides and deuterated alkyl sulphates such as trideuteromethyl-sulphateoxoniums [e.g., boric salts of trialkyloxoniums, in particular (d9)trimethyloxonium tetrafiuoroborate (D₃C)₃OBF₄)] can be used. Methanesulfonyl fluoride (CHF₂SO₂F) or deuterated methanesulfonyl fluoride (CDF₂SO₂F) could also be used to provide the products containing fluorine atoms or deuterium and fluorine atoms. The deuterated/fluorinated alkylating agent is used in the presence of one or more strong bases in anhydrous medium like alkali metal hydrides such as sodium or potassium hydride, alkali metal alkoxides such as potassium tert-butoxide and silver oxide Ag₂O, and 1,8-bis(dimethylamino)naphthalene. Preferably, the reaction is carried out in an organic solvent which is inert under the reaction conditions.

Following the alkylation step, compounds of the invention can be provided by carrying out an esterification step, which can be performed in a known manner. For example, the esterification step can be perfoinied according to the processes described in EP 617,018, WO 96/30355 and in U.S. Provisional Patent Application assigned U.S. Ser. No. 61/426,177 (entitled “Non-Ring Hydroxy Substituted Taxanes and Methods for Synthesizing the Same” filed on Dec. 22, 2010) and the international application of the same title and filed on Dec. 22, 2011, that claims priority to that provisional application. Schematically, an example of an esterification step is shown below.

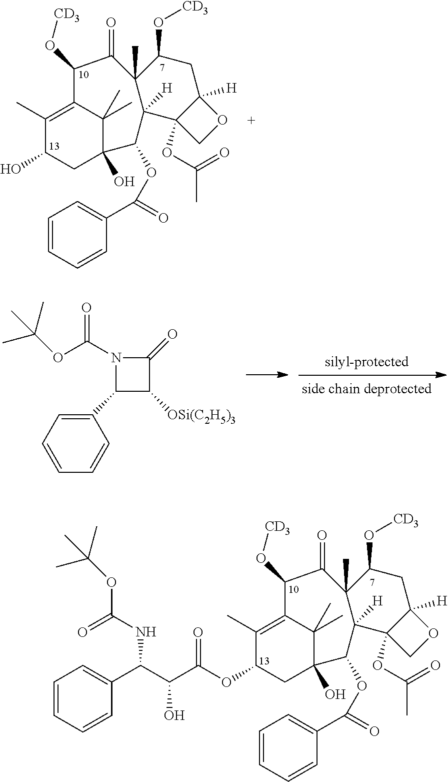

As shown in the schematic immediately above, this second step of the synthesis starts with the 10-deacetylbaccatin modified in positions 7 and 10, which is then coupled in position 13 with a suitably protected β-lactam in the presence of an activating agent, typically chosen from tertiary amines and metallic bases, to form an alkoxide in position 13. The side silyl chain of the intermediate is then deprotected by any one of art-known methods, including the action of an inorganic or organic acid like hydrofluoric acid, trifluoroacetic acid, or various fluoride salts in an appropriate solvent, including a polymer-bound ammonium fluoride.

Alternatively, in a variation of this esterification step, a deuterated/fluorinated reagent which is a protected β-lactam reagent containing a t-Boc group containing deuterium atoms instead of hydrogen atoms can be used. Schematically, this variation of the esterification step is shown below.

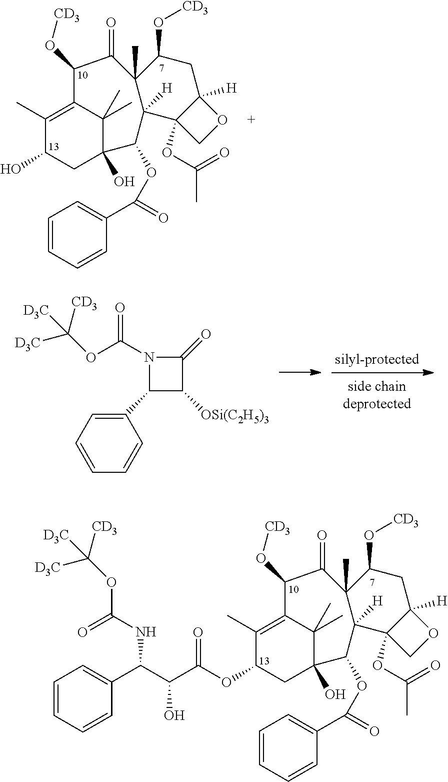

Still other approaches for adding t-Boc groups are described in U.S. Provisional Patent Application assigned U.S. Ser. No. 61/426,177 (entitled “Non-Ring Hydroxy Substituted Taxanes and Methods for Synthesizing the Same” filed on Dec. 22, 2010) and the international application of the same title and filed on Dec. 22, 2011, that claims priority to that provisional application, which approaches can be used or adapted for synthesizing the inventive compounds described herein.

The compounds of the invention (including those encompassed by Formulae I and Ia) require no further modifications. In one more or more embodiments of the invention, compounds are provided wherein the non-ring hydroxy (sometimes referred to as the 2′ hydroxy group) is protected with a hydroxy protecting group. Such non-ring hydroxy protected forms of the compounds provided herein are useful in preparing polymer conjugates. See, for example, U.S. Provisional Patent Application assigned U.S. Ser. No. 61/426,227 (entitled “Multi-arm Polymeric Prodrug Conjugates of Taxane-Based Compounds” filed on Dec. 22, 2010) and the international application of the same title and filed on Dec. 22, 2011, that claims priority to that provisional application.

In one approach for providing protected forms of the non-ring hydroxy group of the inventive compounds provided herein, a condensation reaction can be used to provide the deuterated form. Initially, the deuterated reagent (shown immediately below as a deuterated t-Boc reagent) is added.

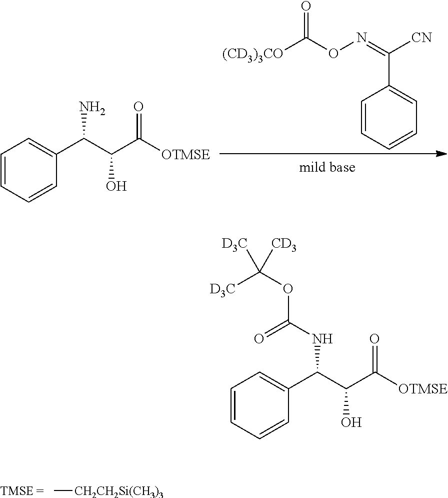

An approach similar to that above is described in U.S. Provisional Patent Application assigned U.S. Ser. No. 61/426,177 (entitled “Non-Ring Hydroxy Substituted Taxanes and Methods for Synthesizing the Same” filed on Dec. 22, 2010) and the international application of the same title and filed on Dec. 22, 2011, that claims priority to that provisional application, and the principles of the reaction described therein apply here as well.

Then, a hydroxy protecting group is added that can be used to ultimately facilitate making a prodrug, e.g., with a protected amino acid as the ester:

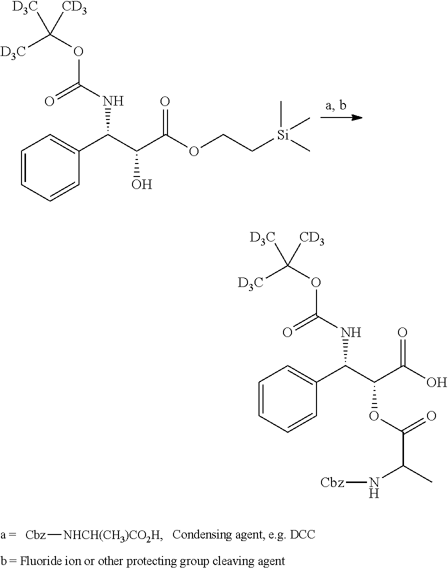

Finally, this component can be attached to the Baccatin III moiety, protected with 7- and 10-hydroxyl groups protected as carbobenzyloxy groups, as described in U.S. Pat. No. 5,688,977, which, like the Cbz on the amine, are removed by hydrogenolysis:

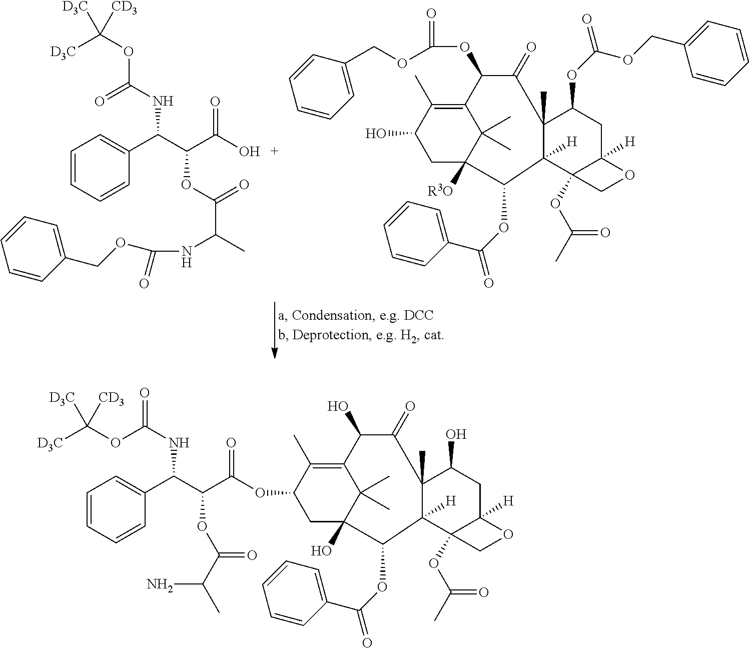

In another exemplary approach for preparing compounds in which 10-deacetylbaccatin III is used begins with first protecting the “7 position.” Several synthetic methods are available to protect the “7 position,” including the approach described in Tetrahedron Letters 35:5543-5546 (1994) and schematically provided below (where the “7 position” is protected with a silyl protecting group):

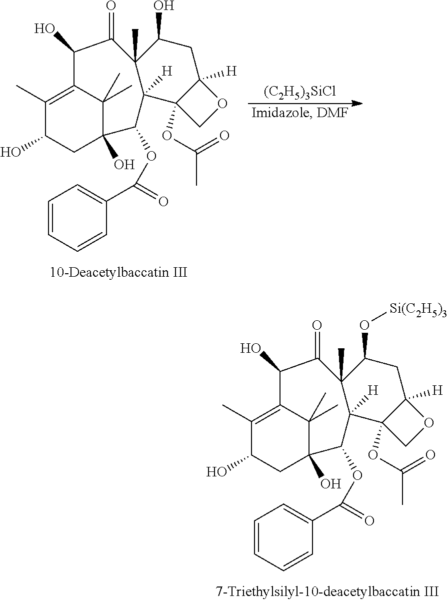

Thereafter, the protected 10-deacetylbaccatin III compound can be alkylated with a deuterated/fluorinated reagent, in a manner described above and schematically shown below:

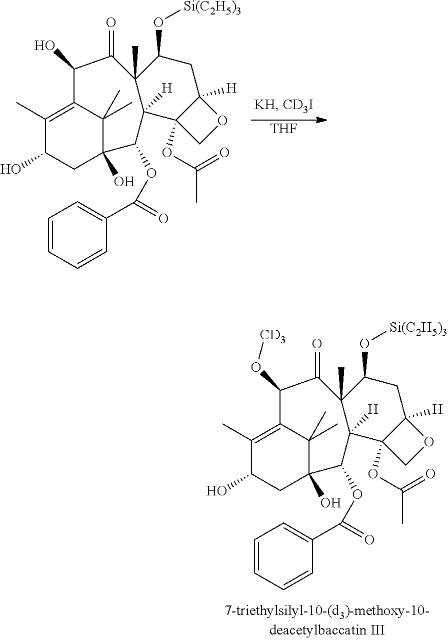

Thereafter, the 7-protected form of 10-deacetylbaccatin III can be esterified in a manner described above, followed by deprotection, an example of which is depicted in the schematic below.

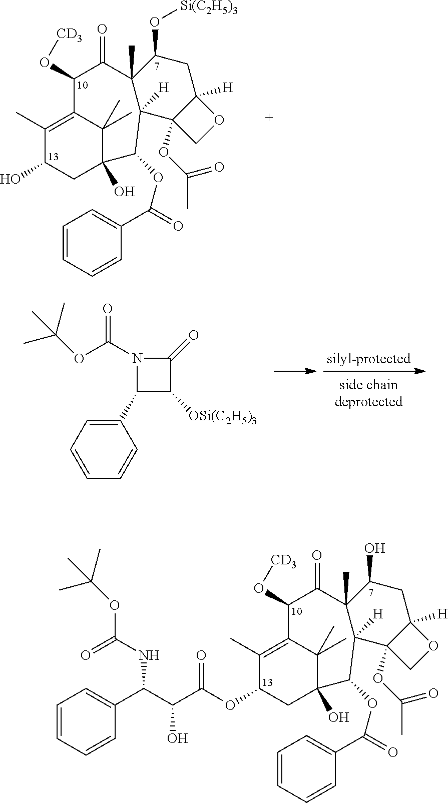

In another exemplary approach for preparing compounds in which 10-deacetylbaccatin III is used begins with first protecting the “10 position.” Several synthetic methods may be used to protect the “10 position.” One such approach is schematically provided below (where the “10 position” is protected with a triethylsilyl protecting group):

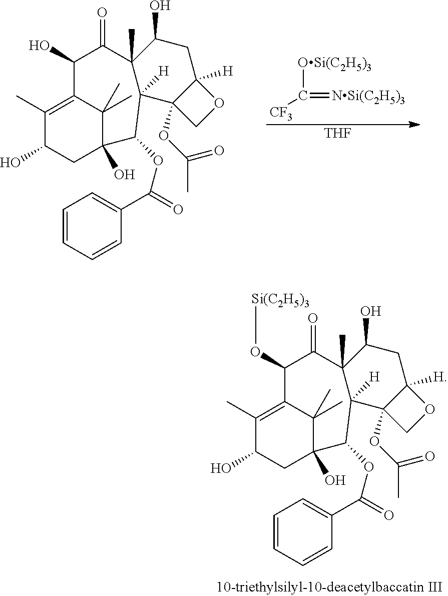

Thereafter, the protected 10-deacetylbaccatin III compound can be alkylated at the 7-position with a deuterated/fluorinated reagent, in a manner described above and schematically shown below:

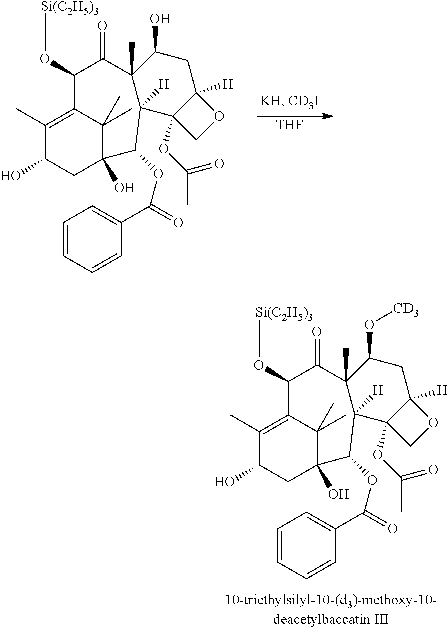

Thereafter, the “10 protected” form of 10-deacetylbaccatin III can be esterified in a manner described above, followed by deprotection, an example of which is depicted in the schematic below:

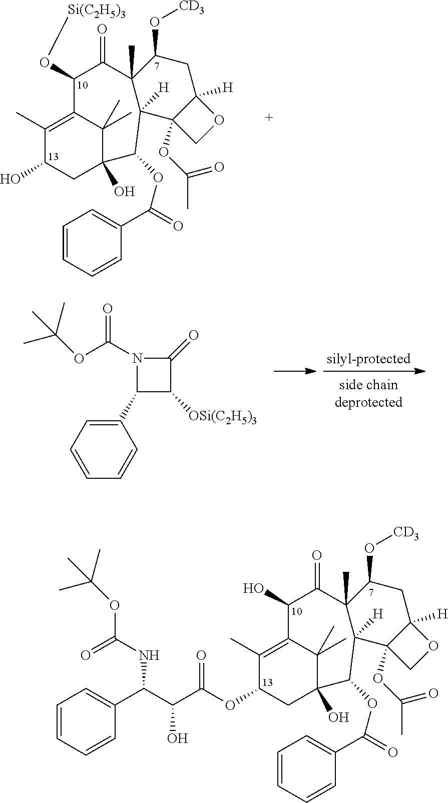

An exemplary approach for preparing compounds of the invention in which a deuterated/fluorinated reagent is used includes acylating a compound of Formula IIa

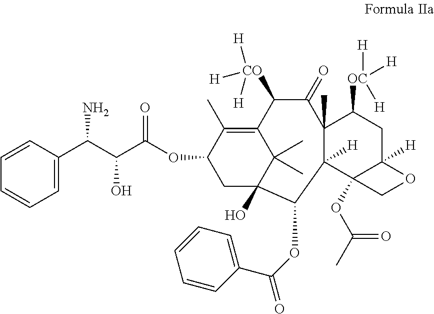

with a deuterated/fluorinated reagent of Formula III,

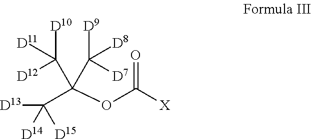

wherein:
X is a leaving group such as benzotriazyl carbonate, halo (e.g., fluoro or chloro);
D⁷ is selected from the group consisting of H, D and F;
D⁸ is selected from the group consisting of H, D and F;
D⁹ is selected from the group consisting of H, D and F;
D¹⁰ is selected from the group consisting of H, D and F;
D¹¹ is selected from the group consisting of H, D and F;
D¹² is selected from the group consisting of H, D and F;
D¹³ is selected from the group consisting of H, D and F;
D¹⁴ is selected from the group consisting of H, D and F; and
D¹⁵ is selected from the group consisting of H, D and F,
and further wherein at least one of D⁷, D⁸, D⁹, D¹⁰, D¹¹, D¹², D¹³, D¹⁴ and D¹⁵ is not H, to form a compound of Formula IV

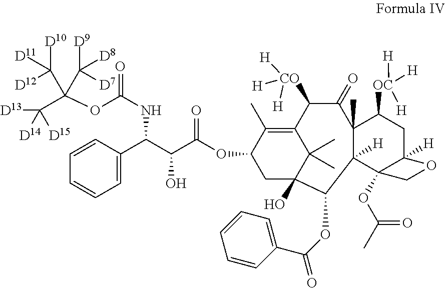

wherein each of D⁷, D⁸, D⁹, D¹⁰, D¹¹, D¹², D¹³, D¹⁴ and D¹⁵ is as defined with respect to Formula III. Compounds of Formula II are described in U.S. Pat. No. 5,847,170.

In addition, compounds of Formula II can be prepared by removing the t-Boc group from the taxane molecule using known methods. One exemplary approach involving the use of trifluoroacetic acid in the presence of methylene chloride is schematically shown immediately below.

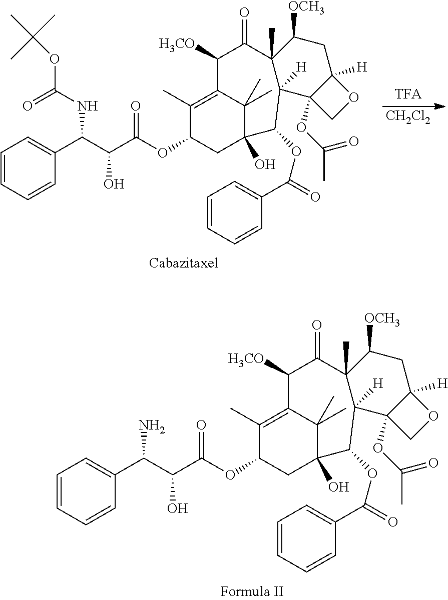

Using a similar approach, the t-Boc group from docetaxel can be removed.

Exemplary acylation conditions to prepare compounds of Formula IV include use of an inert organic solvent in the presence of an inorganic base such as sodium bicarbonate or an organic base such as triethylamine. Exemplary inert organic solvents include esters such as ethyl acetate, isopropyl acetate or n-butyl acetate and halogenated aliphatic hydrocarbons such as dichloromethane or 1,2-dichloroethane. The acylation reaction is performed at a temperature of from about 0° C. to about 50° C. (e.g., about 20° C.).

Exemplary compounds of Formula III include many compounds and the invention is not limited in this regard. One such exemplary deuterated reagent is 2-(ter-[D9]butoxycarbonyloxyimino)-2-phenylacetonitrile, as shown below.

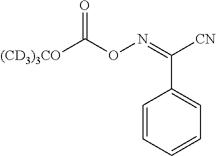

### 2-(tert-[D₉]butoxycarbonyloxyimino)-2-phenylacetonitrile

An exemplary fluorinated compound 2-(1,1,1-trifluoromethyl-2-propoxycarbonyloxyimino)-2-phenylacetonitrile could be used as a fluorination reagent.

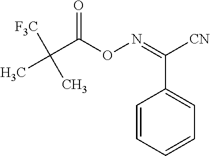 [SMILES: CC(C)(C(=O)O/N=C(/C#N)c1ccccc1)C(F)(F)F]

### 2-(1,1,1-trifluoromethyl-2-propoxycarbonyloxyimino)-2-phenylacetonitrile

Using a similar approach, compounds of the invention having deuterated and/or fluorinated substitutions at both or either one of the two ring-bearing alkoxy substituents can be prepared wherein a compound of Formula IIa is replaced with a compound of Formula IIb,

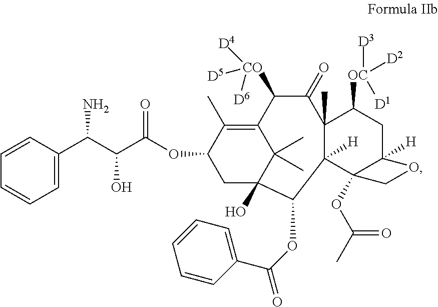

wherein:
D¹ is selected from the group consisting of H, D, F and CF₃;
D² is selected from the group consisting of H, D, F and CF₃;
D³ is selected from the group consisting of H, D, F and CF₃;
D⁴ is selected from the group consisting of H, D, F and CF₃;
D⁵ is selected from the group consisting of H, D, F and CF₃;
D⁶ is selected from the group consisting of H, D, F and CF₃;
   and further wherein at least one of D′, D², D³, D⁴, D⁵ and D⁶ is not H,
   and pharmaceutically acceptable salts and solvates thereof.

Another exemplary approach for preparing compounds of the invention in which a deuterated/fluorinated reagent is used includes starting with a compound of Formula V,

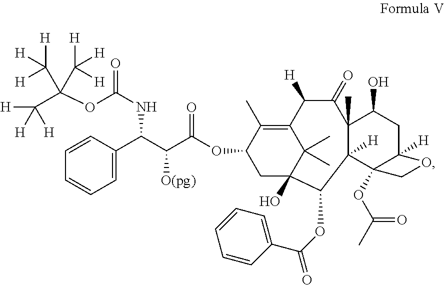

wherein (pg) is a protecting group or H. Compounds of Formula V are described in U.S. Pat. Nos. 5,847,170 and 5,476,954, European Patent 0 336 841, and International Patent Publication WO 94/07878.

Compounds of Formula V are first silylated with a compound of Formula VI,

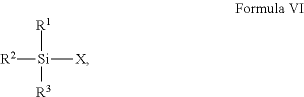

wherein:
X is selected from the group consisting of halo, OR⁴, OS(O)₂CF₃, NMe₂, and O(C═O)R⁵;
R¹ is selected from the group consisting of C₁₋₆ alkyl optionally substituted with a phenyl, C₃₋₆ cycloalkyl and phenyl;
R² is selected from the group consisting of C₁₋₆ alkyl optionally substituted with a phenyl, C₃₋₆ cycloalkyl and phenyl;
R³, is selected from the group consisting of C₁₋₆ alkyl optionally substituted with a phenyl, C₃₋₆ cycloalkyl, styryl and phenyl;
R⁴ is selected from the group consisting of C₁₋₆ alkyl optionally substituted with a phenyl, C₃₋₆ cycloalkyl and phenyl; and
R⁵ is selected from the group consisting of C₁₋₆ alkyl optionally substituted with a phenyl, C₃₋₆ cycloalkyl and phenyl,
   to obtain a compound of Formula VII,

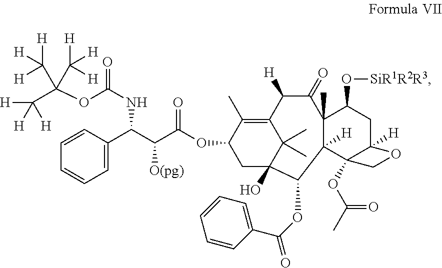

wherein:
(pg) is a protecting group or H;
R¹ is selected from the group consisting of C₁₋₆ alkyl optionally substituted with a phenyl, C₃₋₆ cycloalkyl and phenyl;
R² is selected from the group consisting of C₁₋₆ alkyl optionally substituted with a phenyl, C₃₋₆ cycloalkyl and phenyl;
R³, is selected from the group consisting of C₁₋₆ alkyl optionally substituted with a phenyl, C₃₋₆ cycloalkyl, styryl and phenyl. Compounds of Formula VII are then reacted with a C₁₋₆ deuterated/fluorinated reagent, an exemplary reagent of which is provided in Formula VIII,

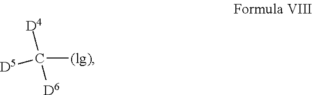

wherein:
D⁴ is selected from the group consisting of H, D, F and CF₃;
D⁵ is selected from the group consisting of H, D, F and CF₃;
D⁶ is selected from the group consisting of H, D, F and CF₃;
   and further wherein at least one of D⁴, D⁵ and D⁶ is not H, to yield a compound of Formula IX,

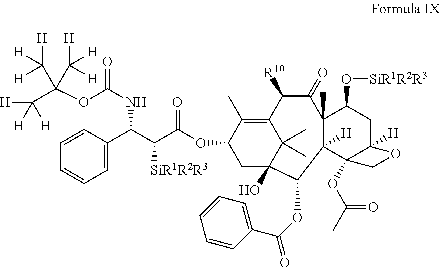

wherein:
each R¹ is independently selected from the group consisting of C₁₋₆ alkyl optionally substituted with a phenyl, C₃₋₆ cycloalkyl and phenyl;
each R² is independently selected from the group consisting of C₁₋₆ alkyl optionally substituted with a phenyl, C₃₋₆ cycloalkyl and phenyl;
each R³ is independently selected from the group consisting of C₁₋₆ alkyl optionally substituted with a phenyl, C₃₋₆ cycloalkyl, styryl and phenyl;
R¹⁰ is a deuterated/fluorinated C₁₋₆ organic radical (e.g.,

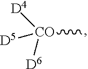

wherein D⁴ is selected from the group consisting of H, D, F and CF₃, D⁵ is selected from the group consisting of H, D, F and CF₃, and D⁶ is selected from the group consisting of H, D, F and CF₃);
and further wherein at least one atom of a compound encompassed by Formula IX is D or F. Preferably a deuterated inert organic solvent and deuterated inorganic base is used if the deuterated/fluorinated reagent of Formula VIII includes one or more deuterium atoms.

Thereafter, compounds of Formula IX are de-silylated and—if the (pg) is present as a protecting group—deprotected by means carrying out conventional deprotection steps to yield a compound of Formula X. In this regard, conventional deprotection steps include, for example, treatment with an acid (such as hydrofluoric acid or trifluoroacetic acid) in the presence of a base such as triethylamine or pyridine optionally substituted with one or more C₁₋₄ alkyl groups, or base bound to a solid support, the base optionally being combined with an inert organic solvent such as a nitrile (e.g., acetonitrile) or a halogenated aliphatic hydrocarbon (e.g., dichloromethane), at a temperature of from 0° to 80° C. If the deuterated/fluorinated reagent of Formula VIII contains one or more deuterium atoms, it is preferred that the acid, base, solvent, etc., are deuterated acids, bases, solvents, etc. With respect to compounds of Formula X, such compounds have the following structure:

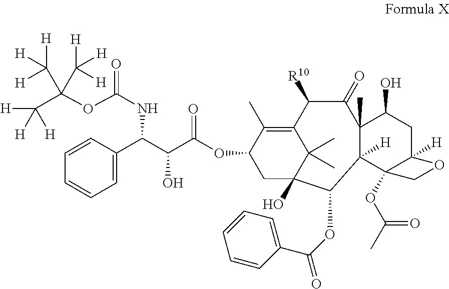

wherein:
R¹⁰ is a deuterated/fluorinated OC₁₋₆ organic radical (e.g.,

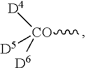

wherein D⁴ is selected from the group consisting of H, D, F and CF₃, D⁵ is selected from the group consisting of H, D, F and CF₃, and D⁶ is selected from the group consisting of H, D, F and CF₃);
and further wherein at least one atom of a compound encompassed by Formula X is D or F, and pharmaceutically acceptable salts and solvates thereof

Using a similar approach, compounds of the invention having deuterated and/or fluorinated substitutions at the tertbutoxycarbonyl substituent can be prepared wherein a compound of Formula IV is substituted for Formula V, to provide a compound of Formula Ia,

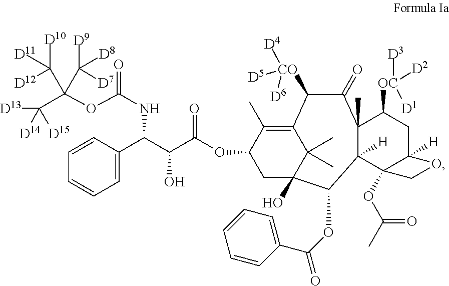

wherein:
D¹ is selected from the group consisting of H, D, F and CF₃;
D² is selected from the group consisting of H, D, F and CF₃;
D³ is selected from the group consisting of H, D, F and CF₃;
D⁴ is selected from the group consisting of H, D, F and CF₃;
D⁵ is selected from the group consisting of H, D, F and CF₃;
D⁶ is selected from the group consisting of H, D, F and CF₃;
D⁷ is selected from the group consisting of H, D and F;
D⁸ is selected from the group consisting of H, D and F;
D⁹ is selected from the group consisting of H, D and F;
D¹⁰ is selected from the group consisting of H, D and F;
D¹¹ is selected from the group consisting of H, D and F;
D¹² is selected from the group consisting of H, D and F;
D¹³ is selected from the group consisting of H, D and F;
D¹⁴ is selected from the group consisting of H, D and F; and
D¹⁵ is selected from the group consisting of H, D and F,
and further wherein at least one of D¹, D², D³, D⁴, D⁵, D⁶, D⁷, D⁸, D⁹, D¹⁰, D¹¹, D¹², D¹³, D¹⁴ and D¹⁵ is not H,
   and pharmaceutically acceptable salts and solvates thereof.

In order to determine the presence of deuterium and/or fluorine atoms in the compounds described herein, conventional techniques for detecting these atoms can be used. For example, IR-spectra can be used to determine the presence of both deuterium and fluorine. An exemplary approach in this regard is described in U.S. Pat. No. 5,895,660, which approach can be adopted with the synthetic methods provided herein. Also, NMR methods (both ¹H and ¹⁹F) can identify the products.

Exemplary compounds of the invention include those selected from the group consisting of

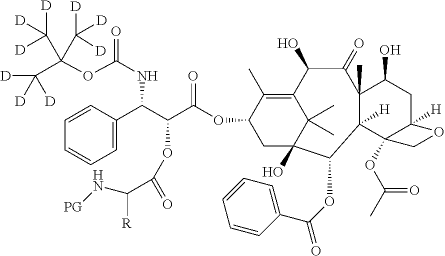

PG=H or Amine protecting group; R═H, Me, lower alkyl or arylalkyl

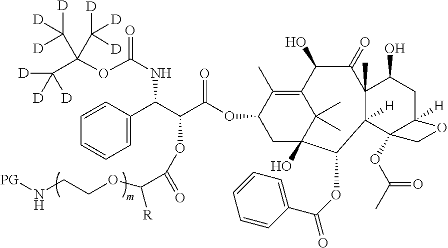

PG=H or Amine protecting group; R═H or Me; m=1-8,

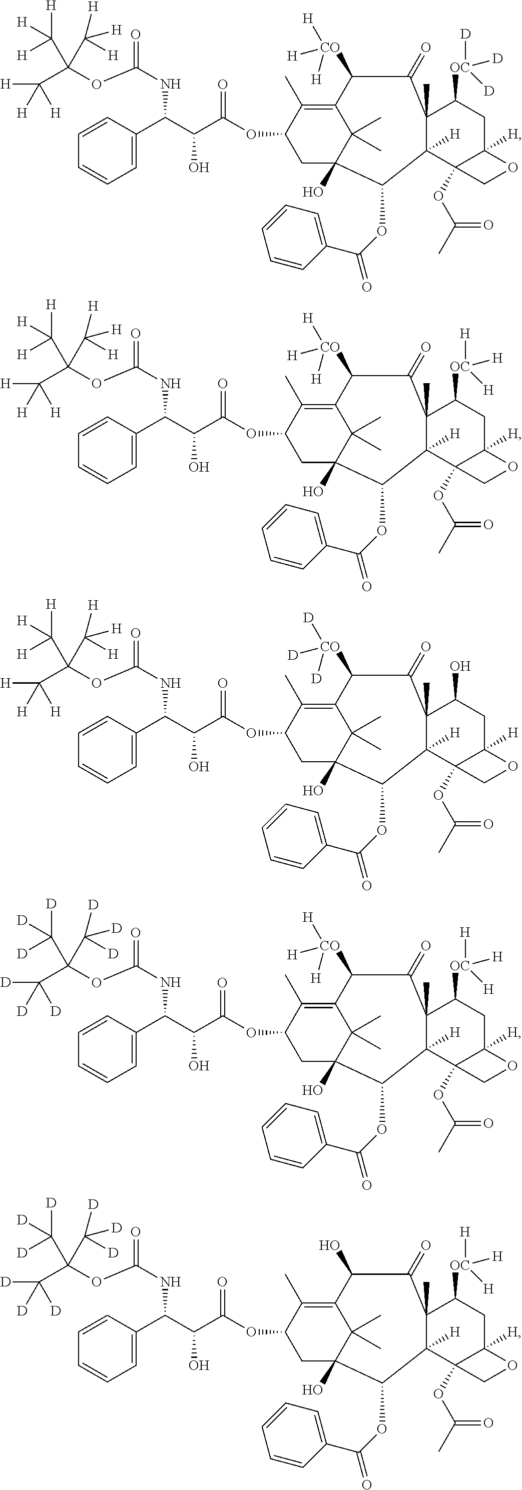

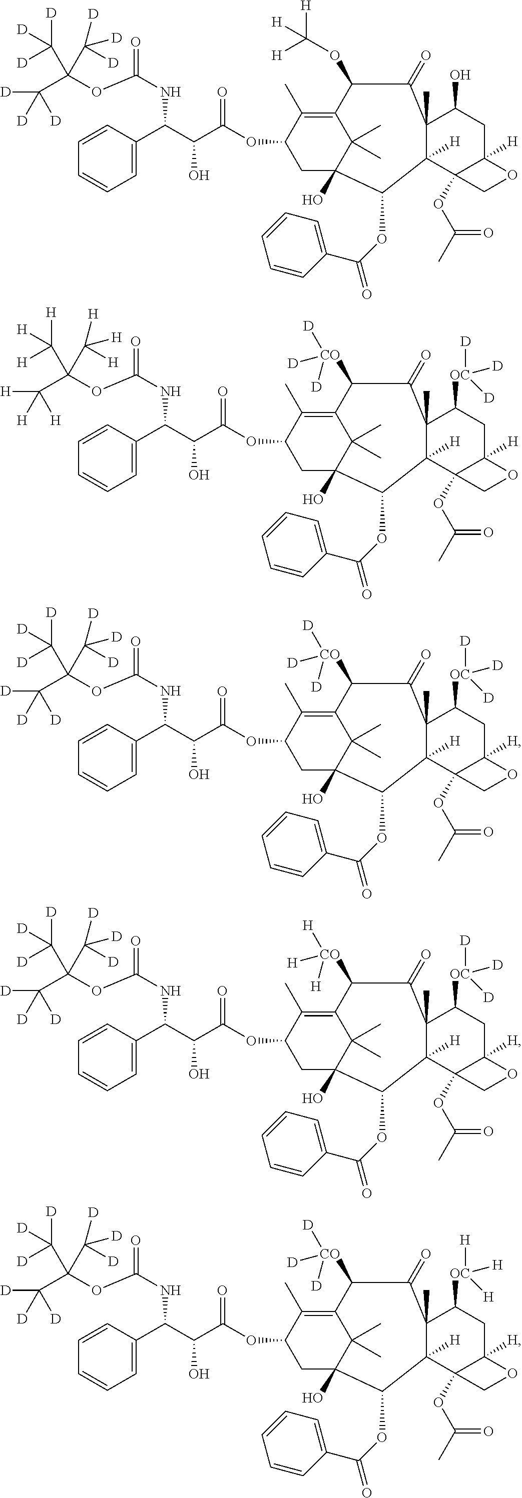

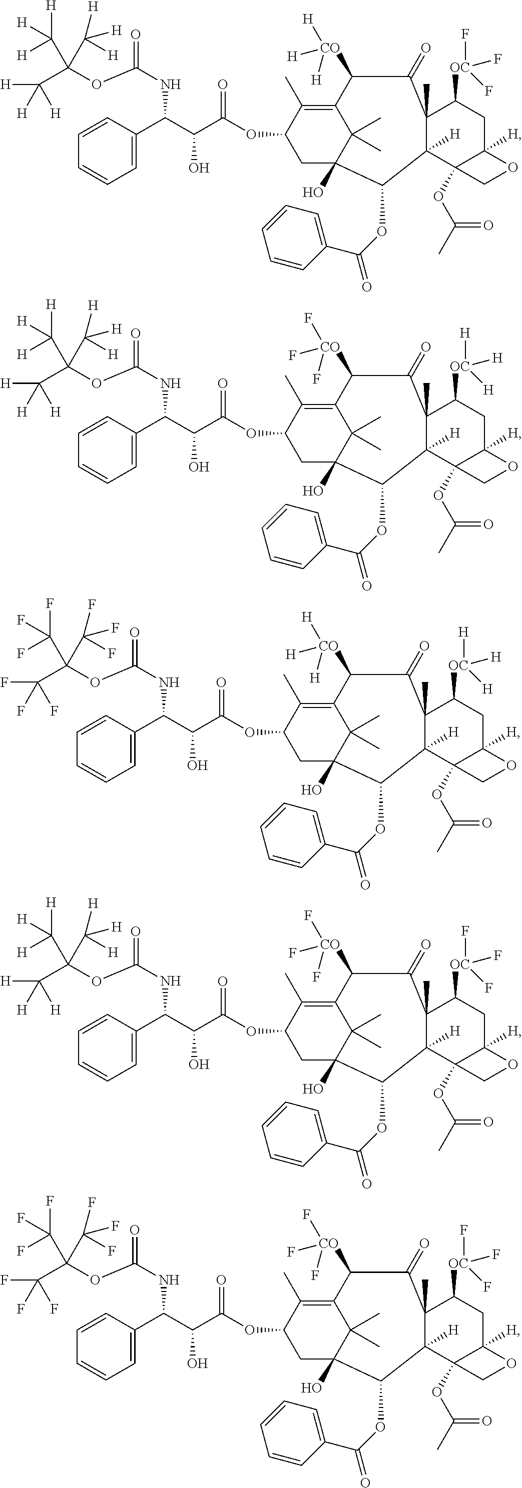

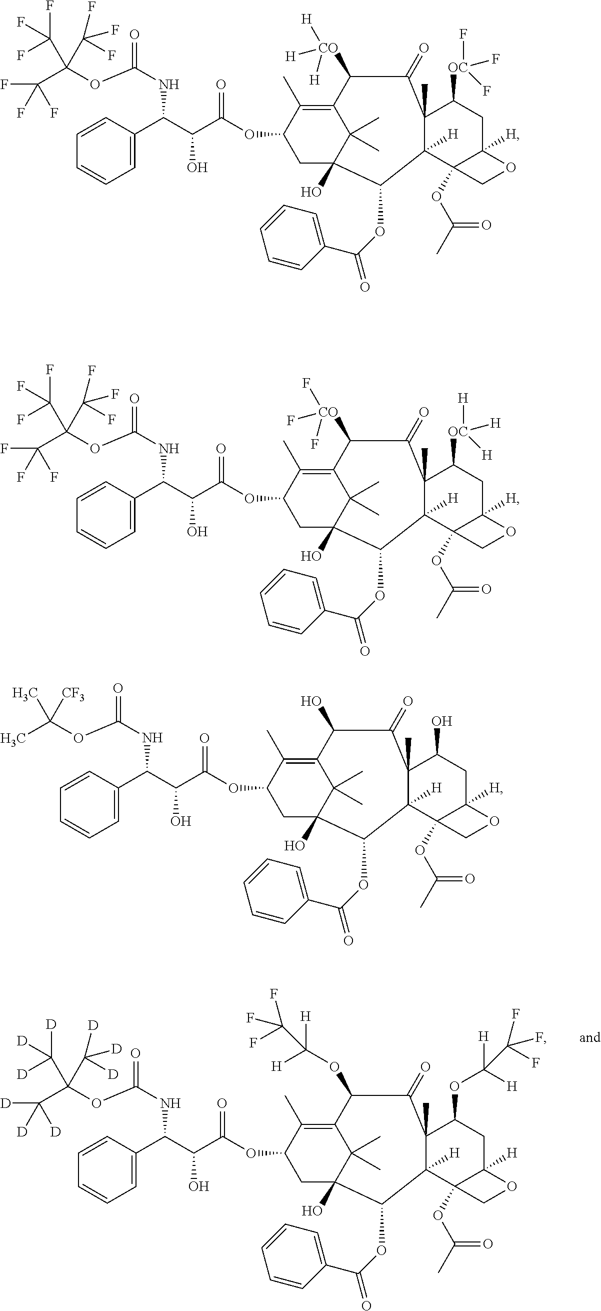

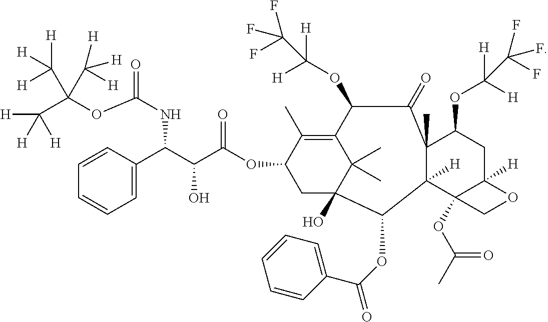

To determine the biological activity of a deuterated and/or fluorinated compound as described herein, it is possible to use conventional assays.

For example, anti-tumor activity against lung cancer can be tested using NCI-H460 lung tumors. Briefly, NCI-H460 lung tumors (30 to 40 fragments of each) can be implanted subcutaneously in the mice (Charles Rivers Labs: NCr nu/nu) near the right axillary area. The day of implantation is designated “Day 0” and the tumors are allowed to reach a weight of 100-245 mg in weight prior to administration of a compound of interest. Animals can be randomized into groups in a manner such that the median tumor weights on the first day of treatment are as close to each other as possible. Mice then receive one or two 2 intravenous doses of test compound or vehicle (saline). Animals are then weighed and the tumors are measured twice weekly after administration of the first injection. The tumor volume is measured by caliper measurements (mm) and using the formula of an ellipsoid sphere: L×W²/2=mm³, where L and W refer to the larger and smaller perpendicular dimensions collected at each measurement. This formula is also used to calculate tumor weight assuming unit density (1 mm³=1 mg). Any animal found moribund or any animal whose tumor reached 4000 mg, ulcerated or is sloughed off is euthanized prior to study termination. By comparing tumor size against saline, it is possible to determine anti-lung tumor activity.

Using a similar approach, it is possible to determine anti-prostate tumor activity by substituting DU-145 prostate tumors for the H460 lung tumors. Other anti-tumor activities (including anti-breast tumor activities) can also be determined in a similar manner.

The compounds of the invention may be administered per se or in the form of a pharmaceutically acceptable salt, and any reference to the compounds of the invention herein is intended to include pharmaceutically acceptable salts. If used, a salt of a compound as described herein should be both pharmacologically and pharmaceutically acceptable, but non-pharmaceutically acceptable salts may conveniently be used to prepare the free active compound or pharmaceutically acceptable salts thereof and are not excluded from the scope of this invention. Such pharmacologically and pharmaceutically acceptable salts can be prepared by reaction of the compound with an organic or inorganic acid, using standard methods detailed in the literature. Examples of useful salts include, but are not limited to, those prepared from the following acids: hydrochloric, hydrobromic, sulfuric, nitric, phosphoric, maleic, acetic, salicyclic, p-toluenesulfonic, tartaric, citric, methanesulfonic, formic, malonic, succinic, naphthalene-2-sulphonic and benzenesulphonic, and the like. Also, pharmaceutically acceptable salts can be prepared as alkaline metal or alkaline earth salts, such as sodium, potassium, or calcium salts of a carboxylic acid group.

The present invention also includes pharmaceutical preparations comprising a compound as provided herein in combination with a pharmaceutical excipient. Generally, the compound itself will be in a solid form (e.g., a precipitate), which can be combined with a suitable pharmaceutical excipient that can be in either solid or liquid form.

Exemplary excipients include, without limitation, those selected from the group consisting of carbohydrates, inorganic salts, antimicrobial agents, antioxidants, surfactants, buffers, acids, bases, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, maltitol, lactitol, xylitol, sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The preparation may also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the preparation as well. Antioxidants are used to prevent oxidation, thereby preventing the deterioration of the conjugate or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant may be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines, fatty acids and fatty esters; steroids, such as cholesterol; and chelating agents, such as EDTA, zinc and other such suitable cations.

Pharmaceutically acceptable acids or bases may be present as an excipient in the preparation. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the compound of the invention in the composition will vary depending on a number of factors, but will optimally be a therapeutically effective dose when the composition is stored in a unit dose container. A therapeutically effective dose can be determined experimentally by repeated administration of increasing amounts of the compound in order to determine which amount produces a clinically desired endpoint.

The amount of any individual excipient in the composition will vary depending on the activity of the excipient and particular needs of the composition. The optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, excipients will be present in the composition in an amount of about 1% to about 99% by weight, preferably from about 5%-98% by weight, more preferably from about 15-95% by weight of the excipient, with concentrations less than 30% by weight most preferred.

These foregoing pharmaceutical excipients along with other excipients and general teachings regarding pharmaceutical compositions are described in “Remington: The Science & Practice of Pharmacy”, 19^{th} Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

The pharmaceutical compositions can take any number of forms and the invention is not limited in this regard. Exemplary preparations are most preferably in a form suitable for oral administration such as a tablet, caplet, capsule, gel cap, troche, dispersion, suspension, solution, elixir, syrup, lozenge, transdermal patch, spray, suppository, and powder.

Oral dosage form is are preferred for those conjugates that are orally active, and include tablets, caplets, capsules, gel caps, suspensions, solutions, elixirs, and syrups, and can also comprise a plurality of granules, beads, powders or pellets that are optionally encapsulated. Such dosage forms are prepared using conventional methods known to those in the field of pharmaceutical formulation and described in the pertinent texts.

Tablets and caplets, for example, can be manufactured using standard tablet processing procedures and equipment. Direct compression and granulation techniques are preferred when preparing tablets or caplets containing the conjugates described herein. In addition to the conjugate, the tablets and caplets will generally contain inactive, pharmaceutically acceptable carrier materials such as binders, lubricants, disintegrants, fillers, stabilizers, surfactants, coloring agents, flow agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethylcellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

Capsules are also preferred oral dosage fauns, in which case the conjugate-containing composition can be encapsulated in the form of a liquid or gel (e.g., in the case of a gel cap) or solid (including particulates such as granules, beads, powders or pellets). Suitable capsules include hard and soft capsules, and are generally made of gelatin, starch, or a cellulosic material. Two-piece hard gelatin capsules are preferably sealed, such as with gelatin bands or the like.

Included are parenteral formulations in the substantially dry form (as a lyophilizate or precipitate, which can be in the form of a powder or cake), as well as formulations prepared for injection, which are liquid and require the step of reconstituting the dry form of parenteral formulation. Examples of suitable diluents for reconstituting solid compositions prior to injection include bacteriostatic water for injection, dextrose 5% in water, phosphate-buffered saline, Ringer's solution, saline, sterile water, deionized water, and combinations thereof.

In some cases, compositions intended for parenteral administration can take the form of nonaqueous solutions, suspensions, or emulsions, normally being sterile. Examples of nonaqueous solvents or vehicles are propylene glycol, polyethylene glycol, vegetable oils, such as olive oil and corn oil, gelatin, and injectable organic esters such as ethyl oleate.

The parenteral formulations described herein can also contain adjuvants such as preserving, wetting, emulsifying, and dispersing agents. The formulations are rendered sterile by incorporation of a sterilizing agent, filtration through a bacteria-retaining filter, irradiation, or heat.

The compounds of the invention can also be administered through the skin using conventional transdermal patch or other transdermal delivery system, wherein the conjugate is contained within a laminated structure that serves as a drug delivery device to be affixed to the skin. In such a structure, the compound is contained in a layer, or “reservoir,” underlying an upper backing layer. The laminated structure can contain a single reservoir, or it can contain multiple reservoirs.

The compounds of the invention can also be formulated into a suppository for rectal administration. With respect to suppositories, the compound is mixed with a suppository base material which is (e.g., an excipient that remains solid at room temperature but softens, melts or dissolves at body temperature) such as cocoa butter (theobroma oil), polyethylene glycols, glycerinated gelatin, fatty acids, and combinations thereof. Suppositories can be prepared by, for example, performing the following steps (not necessarily in the order presented): melting the suppository base material to form a melt; incorporating the compound (either before or after melting of the suppository base material); pouring the melt into a mold; cooling the melt (e.g., placing the melt-containing mold in a room temperature environment) to thereby form suppositories; and removing the suppositories from the mold.

In some embodiments of the invention, the compositions comprising the compounds of the invention may further be incorporated into a suitable delivery vehicle. Such delivery vehicles may provide controlled and/or continuous release of the compounds and may also serve as a targeting moiety. Non-limiting examples of delivery vehicles include, adjuvants, synthetic adjuvants, microcapsules, microparticles, liposomes, and yeast cell wall particles. Yeast cells walls may be variously processed to selectively remove protein component, glucan, or mannan layers, and are referred to as whole glucan particles (WGP), yeast beta-glucan mannan particles (YGMP), yeast glucan particles (YGP), Rhodotorula yeast cell particles (YCP). Yeast cells such as S. cerevisiae and Rhodotorula species are preferred; however, any yeast cell may be used. These yeast cells exhibit different properties in terms of hydrodynamic volume and also differ in the target organ where they may release their contents. The methods of manufacture and characterization of these particles are described in U.S. Pat. Nos. 5,741,495, 4,810,646, 4,992,540, 5,028,703 and 5,607,677, and U.S. Patent Application Publication Nos. 2005/0281781 and 2008/0044438.

The invention also provides a method for administering a compound of the invention as provided herein to a patient suffering from a condition that is responsive to treatment with the compound. The method comprises administering, generally orally, a therapeutically effective amount of the compound (preferably provided as part of a pharmaceutical preparation). Other modes of administration are also contemplated, such as pulmonary, nasal, buccal, rectal, sublingual, transdeimal, and parenteral. As used herein, the term “parenteral” includes subcutaneous, intravenous, intra-arterial, intraperitoneal, intracardiac, intrathecal, and intramuscular injection, as well as infusion injections.

The method of administering may be used to treat any condition that can be remedied or prevented by administration of a particular compound of the invention. Those of ordinary skill in the art appreciate which conditions a specific compound can effectively treat. Exemplary conditions include cancers (e.g., prostate cancer). The actual dose to be administered will vary depend upon the age, weight, and general condition of the subject as well as the severity of the condition being treated, the judgment of the health care professional, and conjugate being administered.

The unit dosage of any given compound of the invention (again, preferably provided as part of a pharmaceutical preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Once the clinical endpoint has been achieved, dosing of the composition is halted.

All articles, books, patents, patent publications and other publications referenced herein are incorporated by reference in their entireties. In the event of an inconsistency between the teachings of this specification and the art incorporated by reference, the meaning of the teachings and definitions in this specification shall prevail (particularly with respect to terms used in the claims appended herein). For example, where the present application and a publication incorporated by reference defines the same term differently, the definition of the term shall be preserved within the teachings of the document from which the definition is located.

### EXPERIMENTAL

It is to be understood that while the invention has been described in conjunction with certain preferred and specific embodiments, the foregoing description as well as the examples that follow are intended to illustrate and not limit the scope of the invention. Other aspects, advantages and modifications within the scope of the invention will be apparent to those skilled in the art to which the invention pertains.

All chemical reagents referred to in the appended examples are commercially available unless otherwise indicated.

### Example 1

### Preparation of 7β,10β-(d₆)-Dimethoxydocetaxel

### Synthesis of 7β,10β-(d₆)-Dimethoxy-10-Deacetylbaccatin III

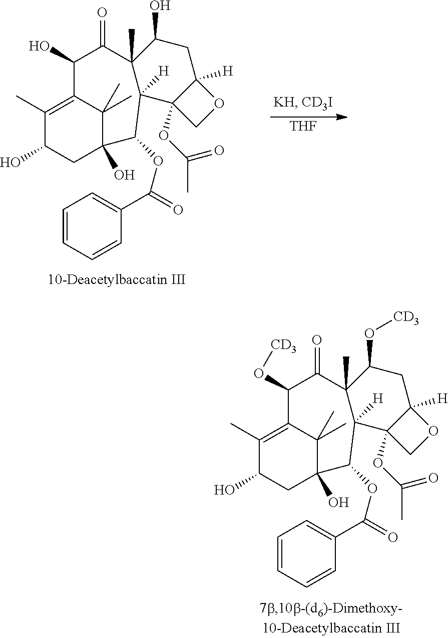

A suspension of 10-deacetylbaccatin III (Sigma-Aldrich; 2.2 g) in tetrahydrofuran (25 ml) and a solution of methyl-(d₃) iodide (9.5 g) in tetrahydrofuran (10 ml) was simultaneously added dropwise to a suspension of potassium hydride (5.0 g), in tetrahydrofuran (15 ml) at −20° C. Next the reaction mixture was stirred for eight hours at room temperature. Then, the reaction mixture was added to water (100 ml) and the resulting mixture was stored overnight at 4° C. Diisopropyl ether (100 ml) was added and the solid precipitate was filtered off. The crude product was purified by silica gel chromatography giving 0.75 g of the desired 7β,10β-(d₆)-dimethoxy-10-deacetylbaccatin III having 98% purity as determined by HPLC analysis.

### Synthesis of 7β,10β-(d₆)-Dimethoxydocetaxel

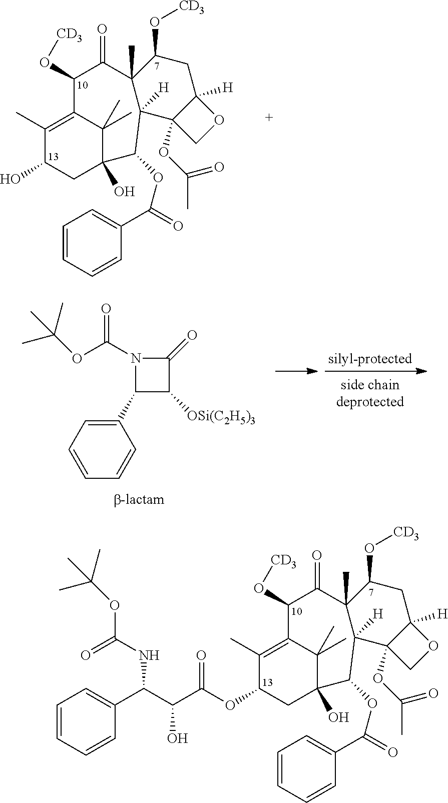

Dicyclohexylcarbodiimide (0.40 g) and then 4-(N,N-dimethylamino)pyridine (0.06 g) were added to a suspension of 7β,10β-(d₆)-dimethoxy-10-deacetylbaccatin III (0.65 g), β-lactam shown above (0.60 g), and powdered 4 A molecular sieves (0.15 g) in 6 ml of ethyl acetate. The mixture was stirred overnight at room temperature under an argon atmosphere, and was concentrated to dryness under reduced pressure. The resulting residue was purified by silica gel chromatography giving the corresponding 2′-triethylsilyl-7β,10β-(d₆)-dimethoxydocetaxel in the form of a white solid (0.55 g).

The product was dissolved in 0.2N solution of hydrogen chloride in ethyl alcohol (40 ml) and stirred overnight at 0° C. under a nitrogen atmosphere. Next, the reaction mixture was diluted with distilled water (15 ml) and the product was extracted two times with dichloromethane (2×60 ml). The extract was dried (MgSO₄) and concentrated to dryness under reduced pressure. The crude product was purified by silica gel chromatography giving 0.45 g of the desired 7β,10β-(d₆)-dimethoxydocetaxel.

### Example 2

### Preparation of 10β-(d₃)-Methoxydocetaxel

### Synthesis of 7β-Triethylsilyl-10-Deacetylbaccatin III

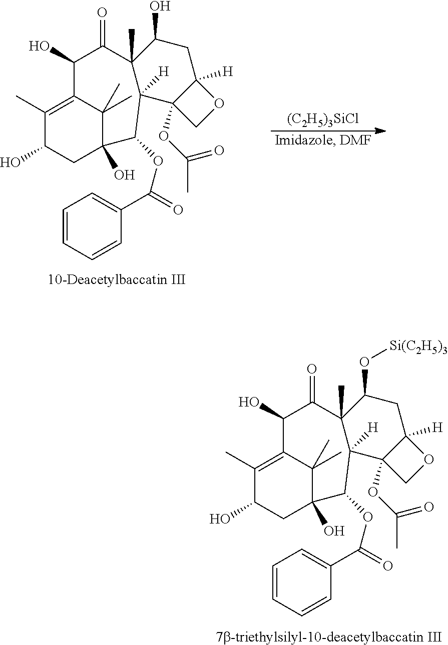

Chlorotriethylsilane (3.7 ml, 0.0221 mol) was added dropwise at 0° C. to a solution of 10-deacetylbaccatin III (3.00 g, 0.0056 mol) and imidazole (1.50 g, 0.0222 mmol) in 140 ml of N,N-dimethylformamide (DMF) and the reaction mixture was stirred for two hours at 0° C. Next, ethyl acetate was added and the obtained solution was washed with water, brine, dried with MgSO₄ and concentrated to dryness. The crude product was purified by silica gel chromatography using hexane:EtOAc=1:1 as an eluent to give 3.35 g of 7β-triethylsilyl-10-deacetylbaccatin III as a white solid.

### Synthesis of 7β-Triethylsilyl,10β-(d₃)-Methoxy-10-Deacetylbaccatin III

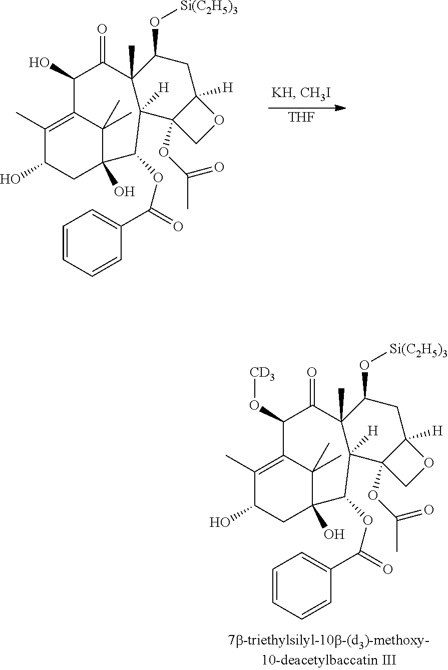

A suspension of 7β-triethylsilyl-10-deacetylbaccatin III (2.7 g) in tetrahydrofuran (25 ml) and a solution of methyl-(d₃) iodide (9.5 g) in tetrahydrofuran (10 ml) was simultaneously added dropwise to a suspension of potassium hydride (5.0 g) in tetrahydrofuran (15 ml) at −20° C. Next the reaction mixture was stirred for eight hours at room temperature. Then, the reaction mixture was added to water (100 ml) and the resulting mixture was stored overnight at 4° C. Diisopropyl ether (100 ml) was added and the solid precipitate was filtered off. The crude product was purified by silica gel chromatography giving 0.85 g of the desired 7β-triethylsilyl-10β-(d₃)-methoxy-10-deacetylbaccatin III having 97% purity as determined by HPLC analysis.

### Synthesis of 10β-(d₃)-Methoxydocetaxel

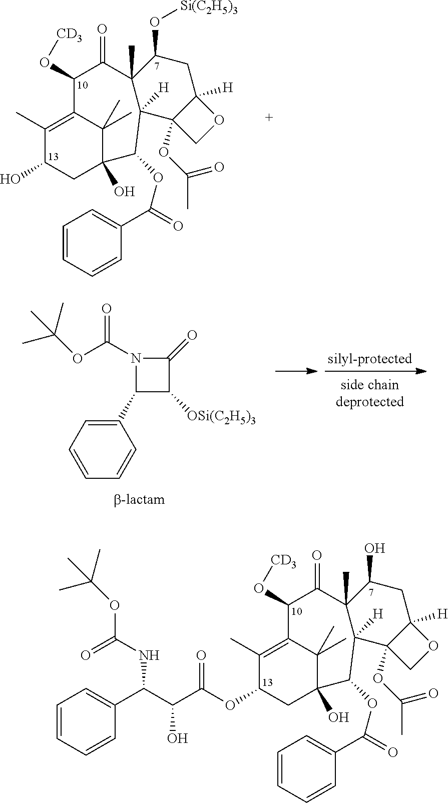

Dicyclohexylcarbodiimide (0.40 g) and then 4-(N,N-dimethylamino)pyridine (0.06 g) were added to a suspension of 7β-triethylsilyl,10β-(d₃)-methoxy-10-deacetylbaccatin III (0.80 g), β-lactam showed above (0.60 g), and powdered 4 A molecular sieves (0.15 g) in 6 ml of ethyl acetate. The mixture was stirred overnight at room temperature under an argon atmosphere, and was concentrated to dryness under reduced pressure. The resulting residue was purified by silica gel chromatography giving 2′-triethylsilyl-7β-triethylsilyl-10β-(d₃)-methoxydocetaxel in the form of a white solid (0.70 g).

The product was dissolved in 0.2N solution of hydrogen chloride in ethyl alcohol (40 ml) and stirred overnight at 0° C. under the nitrogen atmosphere. Next the reaction mixture was diluted with distilled water (15 ml) and the product was extracted two times with dichloromethane (2×60 ml). The extract was dried (MgSO₄) and concentrated to dryness under reduced pressure. The crude product was purified by silica gel chromatography giving 0.52 g of the desired 10β-(d₃)-methoxydocetaxel.

### Example 3

### Preparation of 3′-(1,1,1-Trifluoromethyl-2-Propoxycarbonyloxyimino)-Docetaxel

Removal of Boc Protection from Docetaxel.

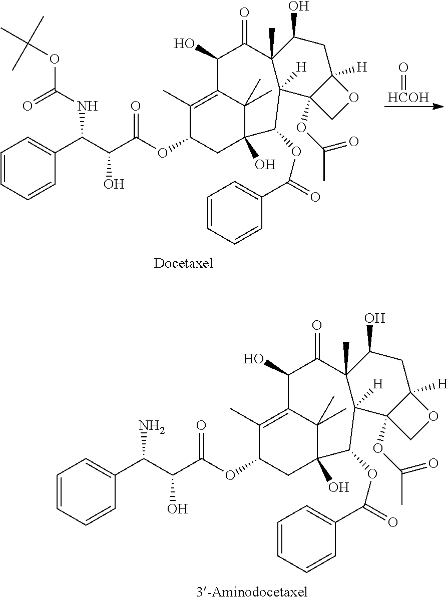

Docetaxel (0.600 g, 0.00074 mol) was dissolved in 50 ml of concentrated formic acid, and the solution was stirred for four hours at room temperature. Next, formic acid was distilled off under reduced pressure. The residue was dissolved in toluene and then toluene was distilled off. This operation was repeated several times to remove residual formic acid. The solid residue was washed with 5% NaHCO₃ solution (2×100 ml), and then the product was extracted with ethyl acetate. The extract was dried (MgSO₄) and the solvent was distilled off under reduced pressure.

The crude product was purified by silica gel chromatography using a mixture EtOAc/MeOH=95/5 mixture as an eluent giving 0.45 g of pure 98.5% pure 3′-aminodocetaxel.

### Synthesis of 3′-(1,1,1-trifluoromethyl-2-propoxycarbonyloxyimino)-docetaxel

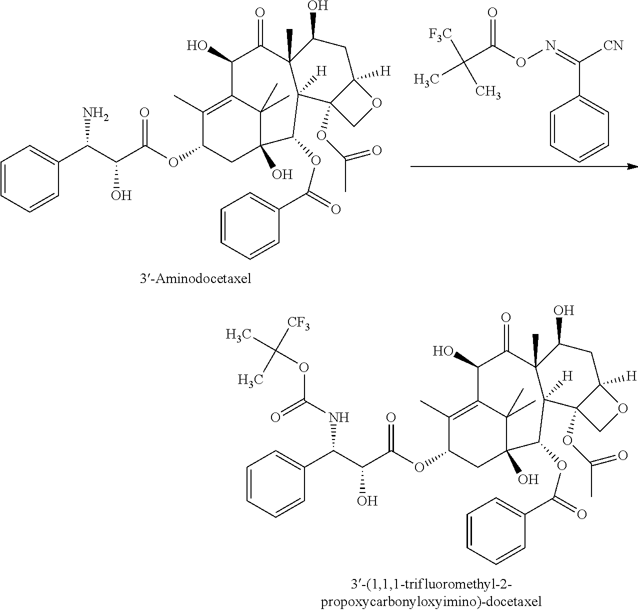

3′-Aminodocetaxel (0.300 g) and 2-(1,1,1-trifluoromethyl-2-propoxycarbonyloxyimino)-2-phenylacetonitrile (0.100 g) were dissolved in pyridine (10 ml). The reaction mixture was stirred overnight at room temperature under nitrogen atmosphere. Next, the solvent was distilled off and the crude compound was purified by silica gel chromatography giving 0.205 g of the desired 3′-(1,1,1-trifluoromethyl-2-propoxycarbonyloxyimino)-docetaxel having purity >98% as determined by RP HPLC.

### Example 4

### Preparation of d₉-Docetaxel

Remove of Boc Protection from Docetaxel

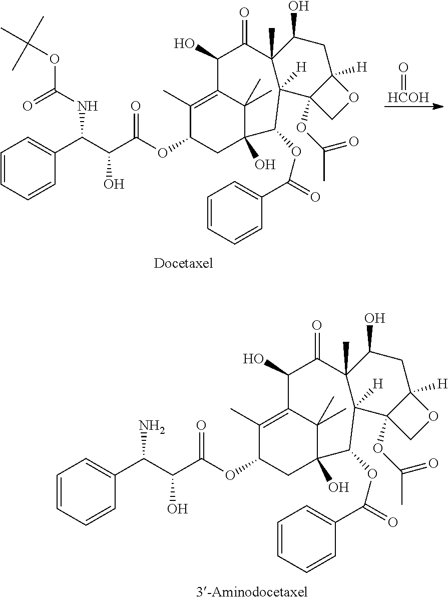

Using a modification of the procedure followed in Example 3 above, docetaxel (10.0 g) was dissolved in 300 ml of concentrated formic acid at ˜5° C. and the solution was stirred at ˜5° C. The reaction progress was monitored by Reversed Phase HPLC. After 4 to 6 hours of the reaction, the solvent was evaporated to dryness under reduced pressure (t max 40° C.). The wet product (the formate salt of the amine) was dried under vacuum overnight and then used in the synthesis of d₉-docetaxel without further purification.

### Synthesis of d9-tert-Butyl Benzotriazolyl Carbonate

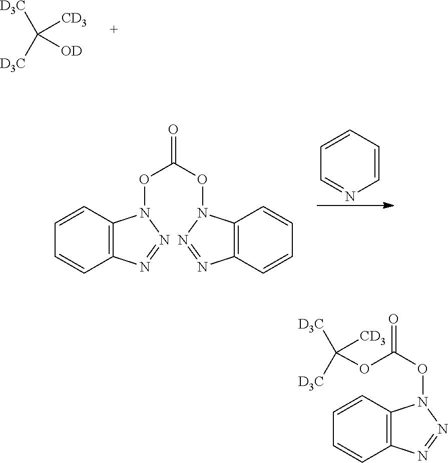

d-10-tert-Butanol (Aldrich; MW=84.08; 13.67 g, 0.1624 mol) was dissolved in 140 ml of anhydrous acetonitrile, followed by addition of dibenzotriazolyl carbonate (DiBTC; MW=296.2; 66.7% dispersion in 1,1,2-trichloroethane; 68.5 g, 0.1542 mol) with 680 ml of anhydrous acetonitrile. The reaction mixture was stirred for 15 minutes, then pyridine (37.8 ml) was added. The reaction mixture was stirred overnight at room temperature under nitrogen atmosphere. The precipitated side products were filtered off. The obtained solution (858 ml) was used directly in the next step of the synthesis. (Calculated concentration of the d9-tert-Butyl Benzotriazolyl Carbonate solution was ˜0.180 mmol/ml.)

Synthesis of d₉-Docetaxel

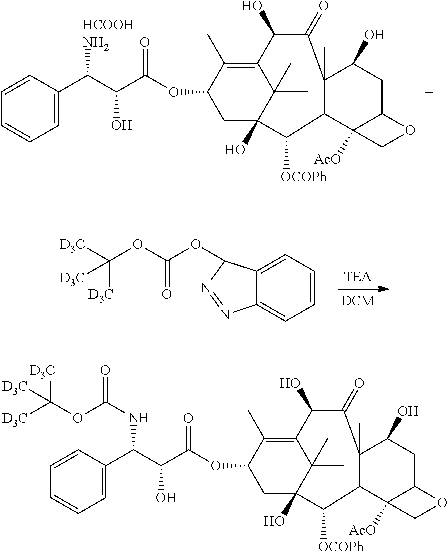

3′-Aminodocetaxel formate salt (0.01238 mol), from above, was dispersed in 200 ml of anhydrous acetonitrile and a solution of d9-tert-Butyl Benzotriazolyl Carbonate (207 ml; 0.0373 mol) was added followed by anhydrous triethylamine (TEA) (8.6 ml, 0.0617 mol; 5.0 fold excess). The reaction mixture was stirred at room temperature for five hours. The solvent was evaporated to dryness at 35-40° C. under reduced pressure. The residue was dissolved in 500 ml of dichloromethane and the solution was washed with 0.1M aq. NaH₂PO₄ (100 ml×2). After drying with MgSO₄, the solution was filtered and the filtrate was concentrated to dryness at 35-40° C. under reduced pressure. The wet product was dried under vacuum for overnight. Next, the product was purified by silica gel chromatography using dichloromethane-ethyl acetate mixture as an eluent. Yield: 4.7 g; HPLC purity ˜97% (UV 254 nm detector) 100% (ELSD detector).

### Example 5

### In vitro Cytotoxicity of Deuterated Docetaxel

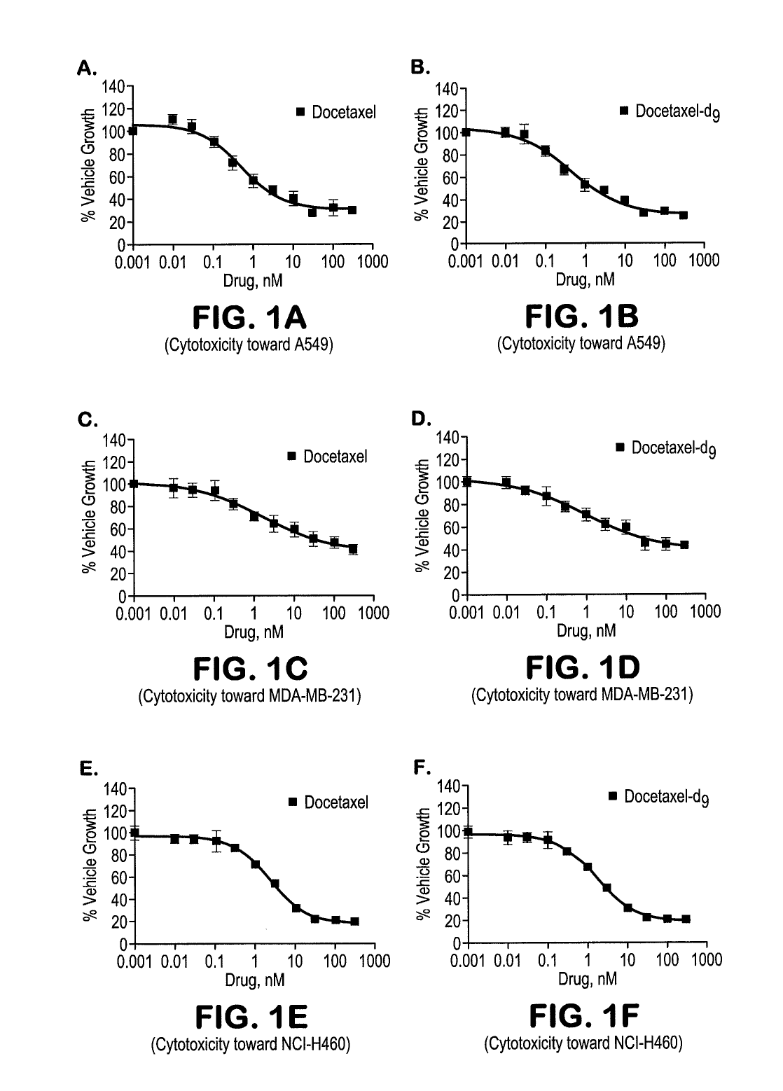

Experimental Procedure: NCI-H460, A549, and MDA-MB-231 cell lines were obtained from ATCC and cultured in RPMI-1640 medium supplemented with 2 mM L-glutamine dipeptide and 10% fetal bovine serum (FBS). For assays, cells were seeded in 96-well plates at 5,000 cells per well in RMPI-1640 medium supplemented with 2 mM L-glutamine dipeptide and 10% FBS. All cells were seeded in a total volume of 50 μL, and placed in a 37° C. humidified 5% CO₂ cell culture incubator overnight. The following day, 50 μL of serial dilutions of 2× stocks of docetaxel and d₉-docetaxel were added to appropriate wells to give the final concentrations. Cell viability was assessed using the CellTiter-Glo Luminescent Cell Viability Assay (Promega) according to the manufacturer's directions. Briefly, plates were removed from the incubator and placed on the bench at room temperature for 30 minutes. Plates were not stacked. Following the 30 minute incubation period at room temperature, 100 μL of CellTiter-Glo reagent were added to each well on the plate and mixed for two minutes, followed by a further ten minutes incubation at room temperature. Luminescence was then recorded using a PerkinElmer Microbeta scintillation and luminescence counter (TriLux). At the same time as drug exposure, a CellTiter-Glo assay was carried out on one plate of each cell line to obtain 0 hr counts for the assay. Following a 72 hour exposure to drugs, a CellTiter-Glo assay was performed on all remaining plates. The results of this study (shown in Table 1 and obtained from the curve fits outlined in FIG. 1A through 1F) suggest that d₉-docetaxel exhibits similar in vitro cytotoxicty as docetaxel.

TABLE 1
IC₅₀ Values of Docetaxel and d₉-Docetaxel Following 72 Hours
of Exposure to A549, MDA-MB-231, and NCI-H460 cell lines.

IC₅₀ (nM)

| Cell Line | Docetaxel | d₉-Docetaxel |
|---|---|---|
| A549 | 1.82 | 1.60 |
| MDA-MB-231 | 35.24 | 25.18 |
| NCI-H460 | 3.52 | 2.68 |

### Example 6

### Maximum Tolerated Dose (MTD) of d₉-Docetaxel in Athymic Mice

Experimental Procedure: MTD for d₉-docetaxel was determined in female athymic nu/nu mice using standard methods. Briefly, 8-12 week old mice were administered d₉-docetaxel intravenously (iv) as shown in Table 2. d₉-Docetaxel solutions were prepared in D5W containing 7.5% Tween 80: 7.5% ethanol and diluted to appropriate concentrations to allow administration volumes of 10 mL/kg. Mice were observed daily for clinical signs and body weights were recorded on days 1-5, 7, 9, 11, 13, and then biweekly until the end of study. All procedures were conducted in compliance with IACUC requirements. Compound dosing was terminated for any group in which mean weight loss exceeded 20% or >10% of animals died. Moribund animals were euthanized and all animals were euthanized at end of study (day 30).

TABLE 2
Maximum Tolerated Dose (MTD) Study Treatment Plan

| Treatment | | Dose of d₉-Docetaxel | | |
|---|---|---|---|---|
| Group | N | (mg/kg) | Route | Schedule |
| 1 | 5 | 10 | iv | qwk x 3 |
| 2 | 5 | 20 | iv | qwk x 3 |
| 3 | 5 | 40 | iv | qwk x 3 |
| 4 | 5 | 7.5 | iv | qwk x 3 |
| 5 | 5 | 15 | iv | qwk x 3 |
| 6 | 5 | 30 | iv | qwk x 3 |

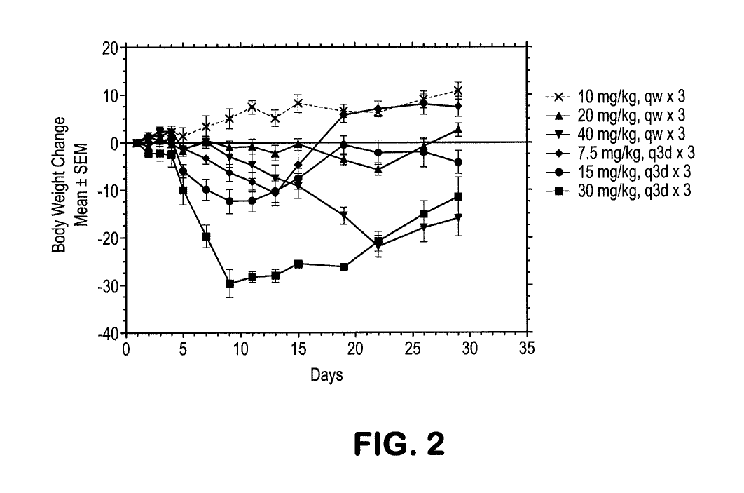

Results: The results are summarized in Table 3 and in FIG. 2. Body weight loss of ˜15% of initial body weight is a typically the maximum desired for tumor xenograft experiments. Based upon these data, it is estimated that the MTD for d₉-docetaxel in athymic nu/nu mice is ˜30 mg/kg when the d₉-docetaxel is administered qwk×3, iv and that the MTD is ˜15 mg/kg when d₉-docetaxel is administered q3d×3, iv.

TABLE 3
Summary of Body Weight Loss Following d₉-Docetaxel Treatment

| | | Dose of | | | Treatment- | |
|---|---|---|---|---|---|---|
| Treatment | | d₉-Docetaxel | BW Nadir | | Related | Mean Day |
| Group | N | (mg/kg) | (Day) | | Deaths | of Death |
| 1 | 5 | 10 | — | | 0 | — |
| 2 | 5 | 20 | −5.6% | (22) | 0 | — |
| 3 | 5 | 40 | −21.9% | (22) | 0 | — |
| 4 | 5 | 7.5 | −10.6% | (13) | 0 | — |
| 5 | 5 | 15 | −12.3% | (9) | 0 | — |
| 6 | 5 | 30 | −29.6% | (9) | 2 | 11.5 |

### Example 7

### In Vitro Metabolic Stability of d₉-Docetaxel

Objective: The object of this example was to compare the metabolic stability of cabazitaxel, docetaxel, and d₉-docetaxel in human liver microsomes in vitro.

Methods: Previously frozen human liver microsomes (mixed gender pool; Xenotech H0630) were thawed in a 37° C. water bath and put on ice immediately after thawing. Cabazitaxel, docetaxel, and d₉-docetaxel were individually incubated with hepatic microsomes for up to thirty minutes (0, 10, 20, and 30 minutes) in a 37° C. water bath with gentle shaking. The final incubation mixture consisted of 1 μM cabazitaxel, docetaxel, or d₉-docetaxel, 2 mM magnesium chloride (Sigma M2670), 4 mM β-nicotinamide adenine dinucleotide phosphate sodium salt (Sigma N3139), 40 mM D-glucose 6-phosphate disodium salt hydrate (Sigma G7250), 0.4 unit/mL of glucose 6-phosphate dehydrogenase (Sigma G6378), and 0.5 mg/mL thawed human liver microsomal proteins or boiled liver microsomal proteins in 100 mM potassium phosphate buffer at pH 7.4. The incubation mixture (500 μL) was prepared in individual silanized tube (CTS-13100) for each time point and for each test article. Incubation was terminated by adding 500 μL cold water:acetonitrile 1:1 (v/v) spiked with 400 nM paclitaxel (internal standard) to the tube. After protein precipitation by centrifugation at 4,000 rpm for 30 minutes at 4° C., 200 μL of supernatant was collected for quantitation of cabazitaxel, docetaxel, and d₉-docetaxel using an AB SCIEX 4000 QTRAP Mass spectrometer. The slope of the linear regression from log percentage remaining versus incubation time relationships (−k) was used in the calculation of intrinsic clearance: Intrinsic clearance (CLᵢₙₜ)=k*(μL incubation/mg of microsomal protein).

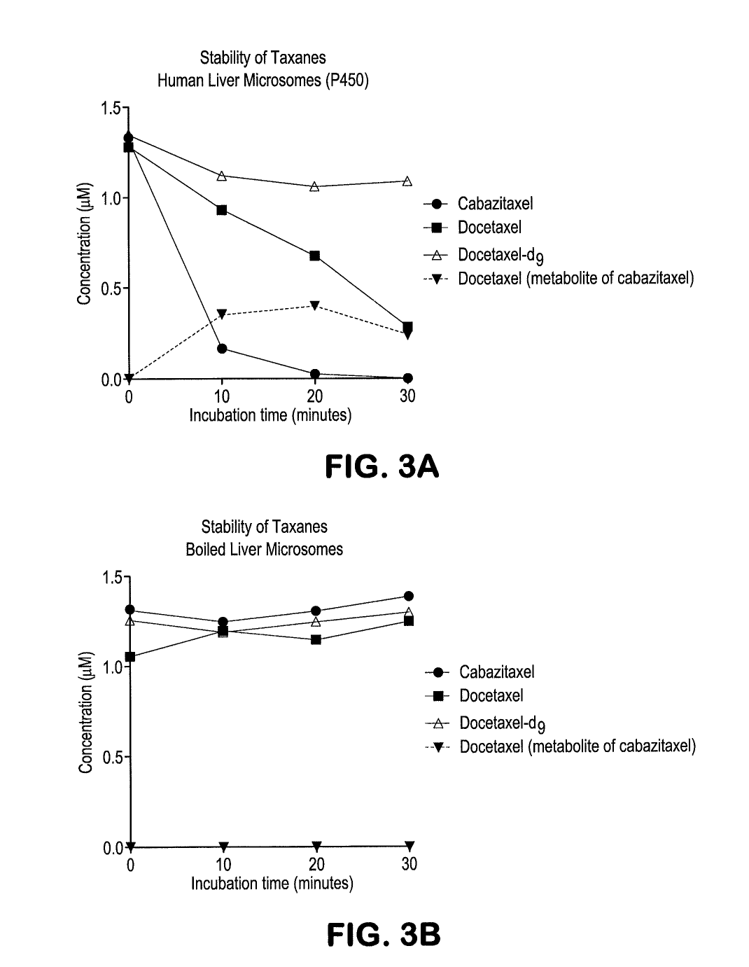

Results: The concentration-time profiles of cabazitaxel, docetaxel, and d₉-docetaxel following incubation with human and boiled liver microsomes are shown in FIG. 3A for human liver microsomes and FIG. 3B for boiled liver microsomes. The intrinsic clearance rate of d₉-docetaxel (13.9 μL/min/mg) was a 29.5- and 7.0-fold lower than that of cabazitaxel (409.8 μL/min/mg) and docetaxel (97.2 μL/min/mg), respectively, in human liver microsomes. The formation of docetaxel, a known metabolite of cabazitaxel, was detected after incubation of cabazitaxel with human liver microsomes and the apparent formation rate of docetaxel from cabazitaxel was 69.5 pmol/mg/min. Cabazitaxel, docetaxel, and d₉-docetaxel were fairly stable during incubation with boiled liver microsomes. Docetaxel formation was not observed after incubation of cabazitaxel with boiled liver microsomes.

1. A compound selected from the group consisting of a deuterated cabazitaxel and a deuterated docetaxel.

2. A compound selected from the group consisting of a fluorinated cabazitaxel and a fluorinated docetaxel.

3. A compound having a structure encompassed by the following formula: 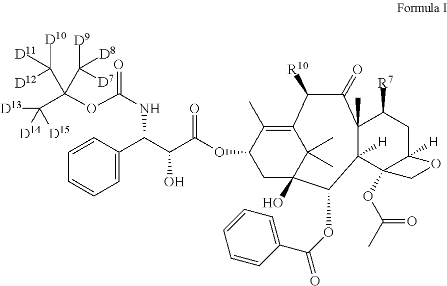
wherein:
R¹⁰ is selected from the group consisting of OH, OC₁₋₆ organic radical, and a deuterated/fluorinated OC₁₋₆ organic radical;
R⁷ is selected from the group consisting of OH, a OC₁₋₆ organic radical, and a deuterated/fluorinated OC₁₋₆ organic radical;
D⁷ is selected from the group consisting of H, D and F;
D⁸ is selected from the group consisting of H, D and F;
D⁹ is selected from the group consisting of H, D and F;
D¹⁰ is selected from the group consisting of H, D and F;
D¹¹ is selected from the group consisting of H, D and F;
D¹² is selected from the group consisting of H, D and F;
D¹³ is selected from the group consisting of H, D and F;
D¹⁴ is selected from the group consisting of H, D and F; and
D¹⁵ is selected from the group consisting of H, D and F,
and further wherein at least one atom in the compound is either D or F,
and pharmaceutically acceptable salts thereof.

4. The compound of claim 3, wherein R¹⁰ is a deuterated/fluorinated OC₁₋₆ organic radical.

5. The compound of claim 4, wherein the deuterated/fluorinated OC₁₋₆ organic radical is 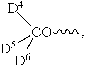
wherein D⁴ is selected from the group consisting of H, D, F and CF₃, D⁵ is selected from the group consisting of H, D, F and CF₃, and D⁶ is selected from the group consisting of H, D, F and CF₃.

6. The compound of claim 1, wherein R⁷ is a deuterated/fluorinated OC₁₋₆ organic radical.

7. The compound of claim 6, wherein the deuterated/fluorinated OC₁₋₆ organic radical is 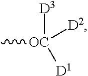
wherein D¹ is selected from the group consisting of H, D, F and CF₃, D² is selected from the group consisting of H, D, F and CF₃, and D³ is selected from the group consisting of H, D, F and CF₃.

8. The compound of claim 3, wherein each of R¹⁰ and R⁷ are H.

9. The compound of claim 3, wherein each of R¹⁰ and R⁷ are CH₃.

10. The compound of claim 3, wherein each of R¹⁰ is CH₃ and R⁷ is H.

11. The compound of claim 3, wherein each of R¹⁰ is H and R⁷ is CH₃.

12. The compound of claim 3, having a structure encompassed by the following formula: 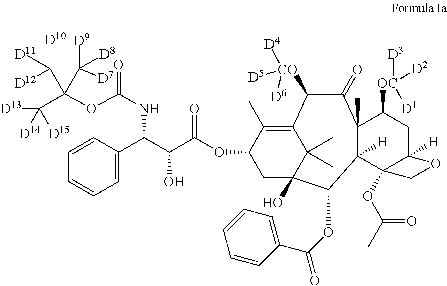
wherein:
D¹ is selected from the group consisting of H, D, F and CF₃;
D² is selected from the group consisting of H, D, F and CF₃;
D³ is selected from the group consisting of H, D, F and CF₃;
D⁴ is selected from the group consisting of H, D, F and CF₃;
D⁵ is selected from the group consisting of H, D, F and CF₃;
D⁶ is selected from the group consisting of H, D, F and CF₃;
D⁷ is selected from the group consisting of H, D and F;
D⁸ is selected from the group consisting of H, D and F;
D⁹ is selected from the group consisting of H, D and F;
D¹⁰ is selected from the group consisting of H, D and F;
D″ is selected from the group consisting of H, D and F;
D¹² is selected from the group consisting of H, D and F;
D¹³ is selected from the group consisting of H, D and F;
D¹⁴ is selected from the group consisting of H, D and F; and
D¹⁵ is selected from the group consisting of H, D and F,
and further wherein at least one of D¹, D², D³, D⁴, D⁵, D⁶, D⁷, D⁸, D⁹, D¹⁰, D¹¹, D¹², D¹³, D¹⁴ and D¹⁵ is not H, and pharmaceutically acceptable salts thereof.

13. The compound of claim 3, selected from the group consisting of

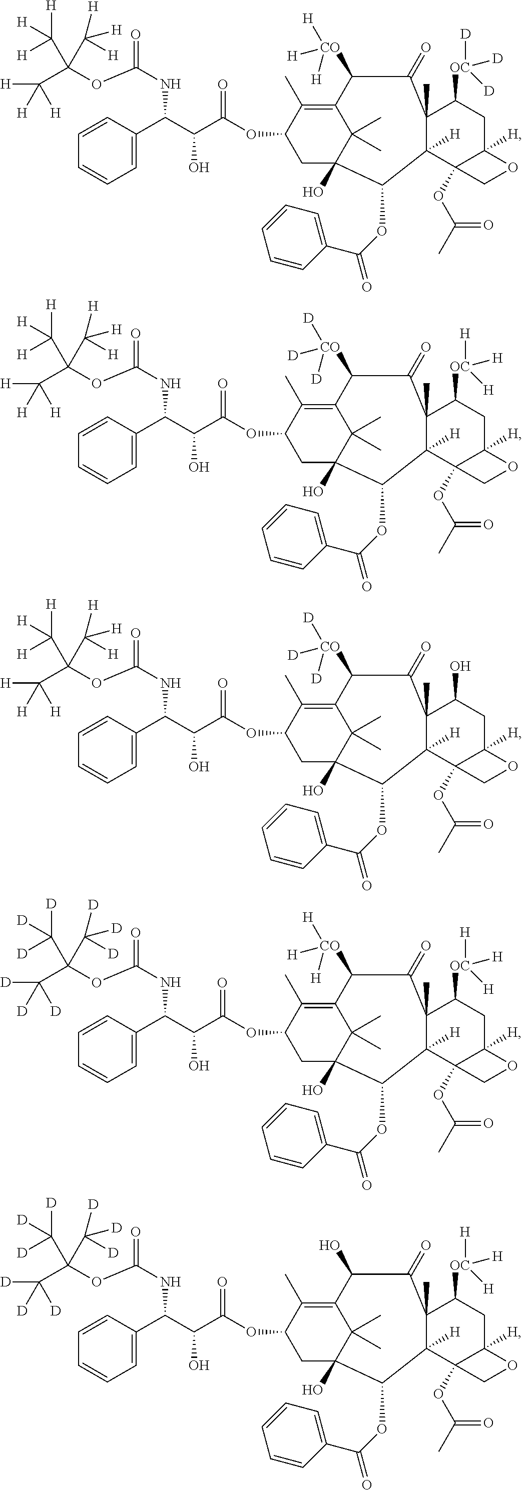

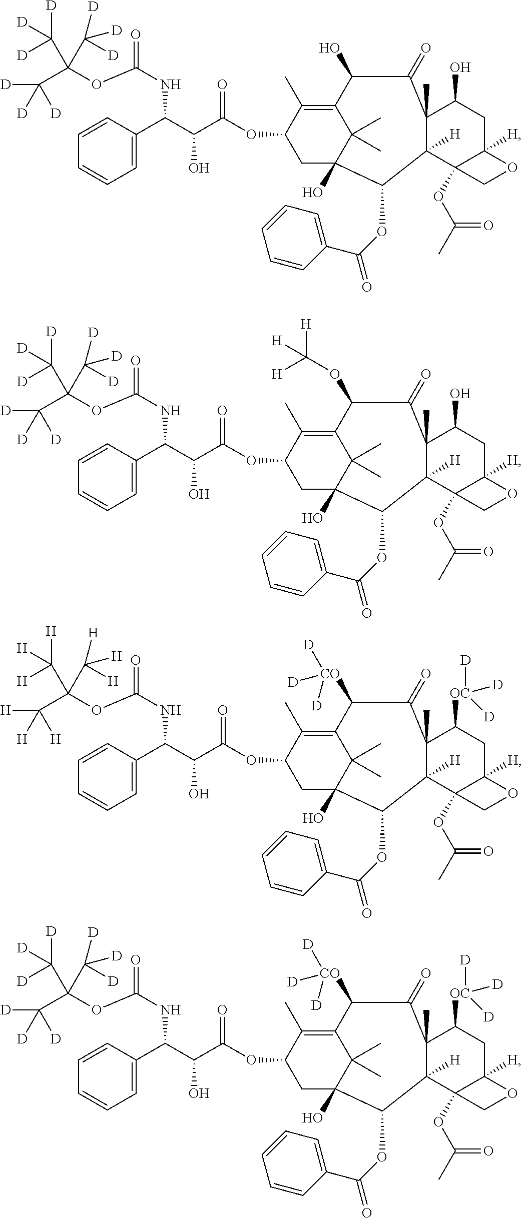

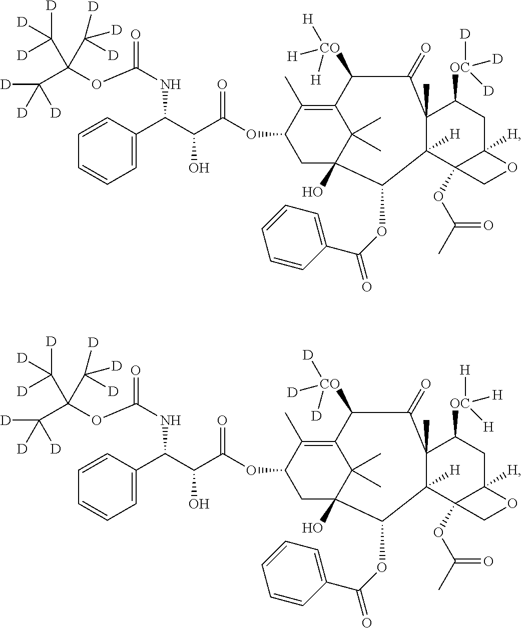

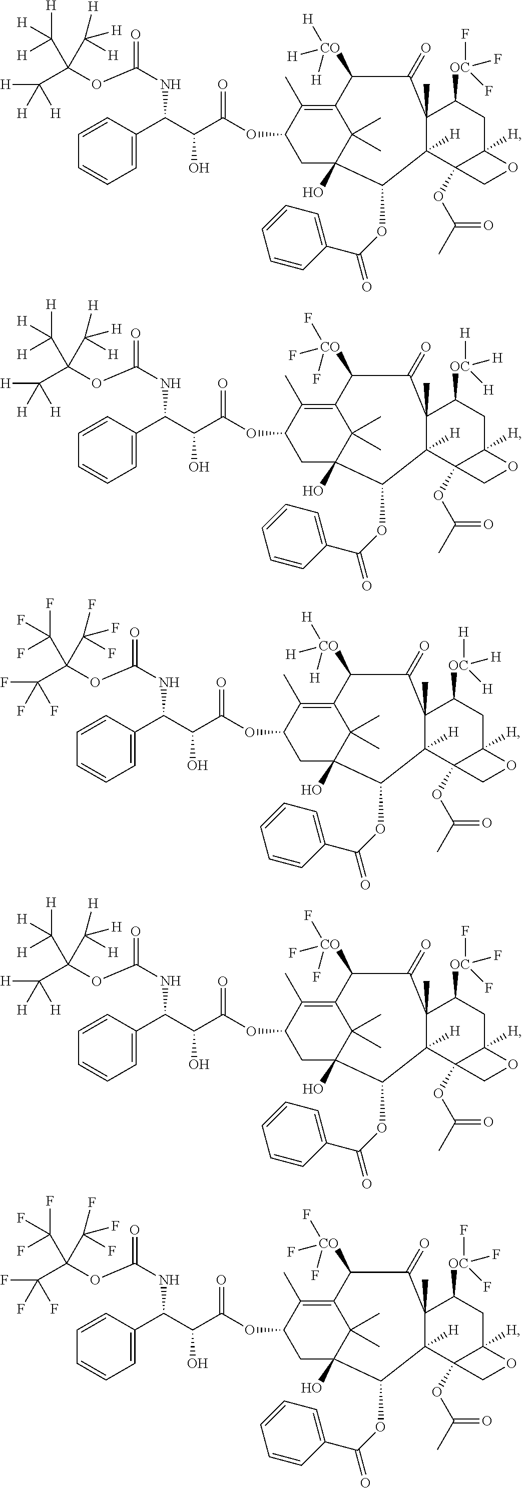

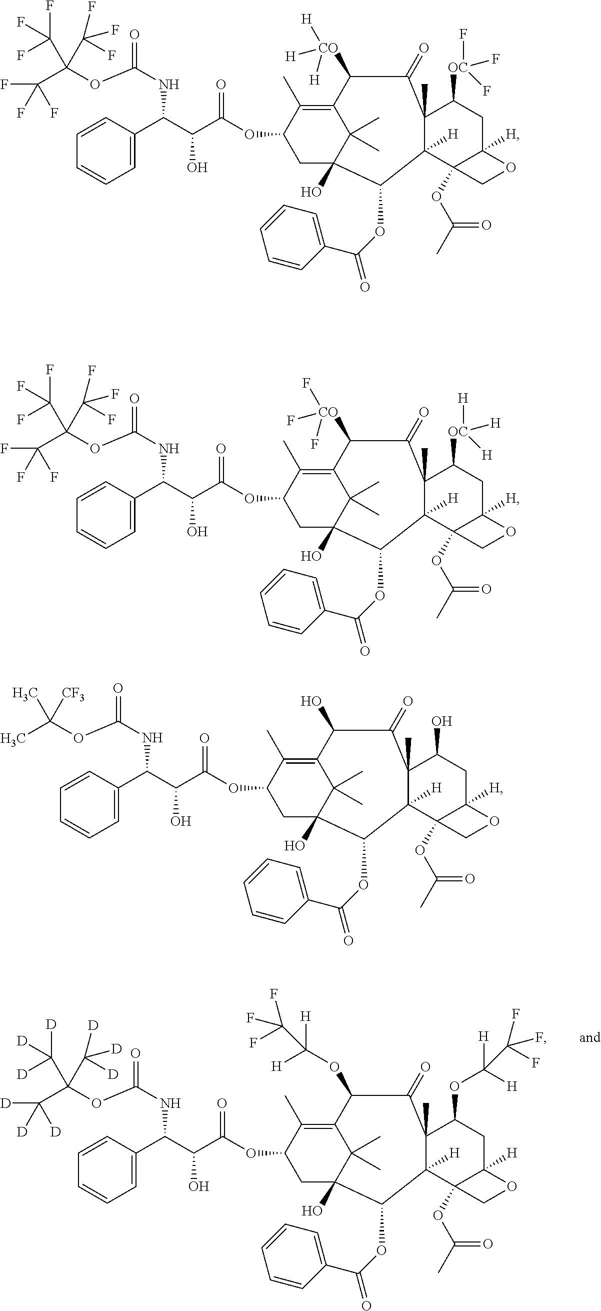

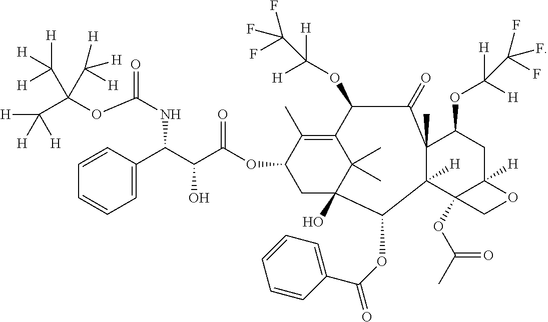

14. The compound of claim 3, having a structure corresponding to:

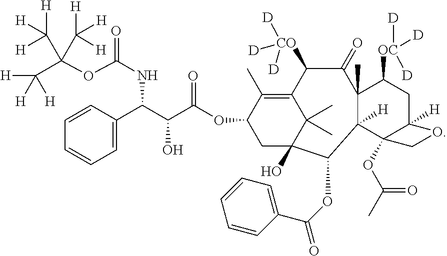

15. The compound of claim 3, having a structure corresponding to:

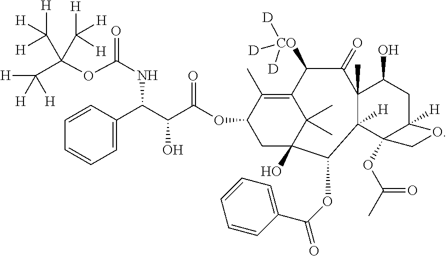

16. The compound of claim 3, having a structure corresponding to:

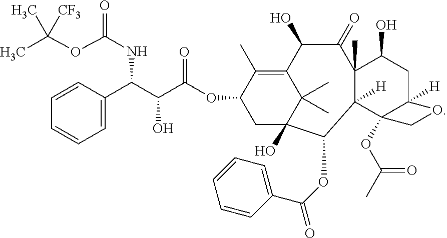

17. The compound of claim 3, having a structure corresponding to:

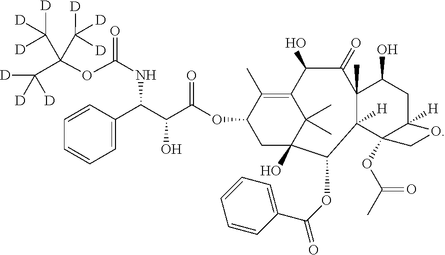

18. A composition comprising a compound of claim 1 and an optional pharmaceutically acceptable excipient.

19. A dosage form comprising a compound of claim 1.

20. A method comprising deuterating a taxane.

21. A method comprising fluorinating a taxane.

22. A method comprising administering a compound of claim 1 to a mammal in need thereof.

23. A compound having a structure encompassed by the following formula: 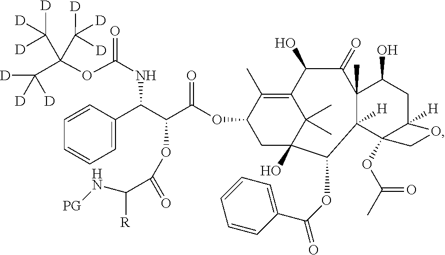
wherein PG is H or an amine protecting group, and R is selected from the group consisting of H, lower alkyl or arylakyl.

24. A compound having a structure encompassed by the following formula: 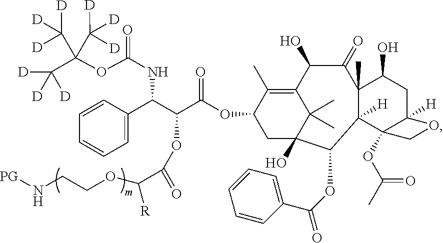
wherein PG is H or an amine protecting group, R is H or lower alkyl, and m is an integer of from 1 to 8, inclusive.
